SEQUENCE LISTING (14 sequences)

SEQ ID NO: 1 - DNA, 19 bp, Artificial Sequence
  Description of Artificial Sequencequantitative PCR forward primer Beta actin F
gaaccctaaggccaacgct

SEQ ID NO: 2 - DNA, 19 bp, Artificial Sequence
  Description of Artificial Sequencequantitative PCR reverse primer Beta actin R
cacgcacgatttccctctc

SEQ ID NO: 3 - DNA, 16 bp, Artificial Sequence
  Description of Artificial Sequencequantitative PCR forward primer histamine type 1 receptor (H1R) F
ttgaaccgagagcgga

SEQ ID NO: 4 - DNA, 17 bp, Artificial Sequence
  Description of Artificial Sequencequantitative PCR reverse primer histamine type 1 receptor (H1R) R
tgcccttaggaacgaat

SEQ ID NO: 5 - DNA, 16 bp, Artificial Sequence
  Description of Artificial Sequencequantitative PCR forward primer histamine type 2 receptor (H2R) F
tggcacggttcattcc

SEQ ID NO: 6 - DNA, 16 bp, Artificial Sequence
  Description of Artificial Sequencequantitative PCR reverse primer histamine type 2 receptor (H2R) R
gcagtagcggtccaag

SEQ ID NO: 7 - DNA, 16 bp, Artificial Sequence
  Description of Artificial Sequencequantitative PCR forward primer platelet activating factor receptor (PAFR) F
ctacaacgagggcgac

SEQ ID NO: 8 - DNA, 17 bp, Artificial Sequence
  Description of Artificial Sequencequantitative PCR reverse primer platelet activating factor receptor (PAFR) R
gggacaaagagatgcca

SEQ ID NO: 9 - DNA, 16 bp, Artificial Sequence
  Description of Artificial Sequencequantitative PCR forward primer lipocalin-type prostaglandin D synthase (PGDS) F
ctggttccgggagaag

SEQ ID NO: 10 - DNA, 18 bp, Artificial Sequence
  Description of Artificial Sequencequantitative PCR reverse primer lipocalin-type prostaglandin D synthase (PGDS) R
agcgtactcgtcatagtt

SEQ ID NO: 11 - DNA, 16 bp, Artificial Sequence
  Description of Artificial Sequencequantitative PCR forward primer mouse mast cell protease-7 (MMCP-7) F
acacgagaaggcattg

SEQ ID NO: 12 - DNA, 17 bp, Artificial Sequence
  Description of Artificial Sequencequantitative PCR reverse primer mouse mast cell protease-7 (MMCP-7) R
aggtactgcttacggag

SEQ ID NO: 13 - protein, 12 aa, Artificial Sequence
  Description of Artificial Sequenceamino- terminal extracellular domain peptide of  histamine type 1 receptor (H1R)
SSASEDKMCEGN

SEQ ID NO: 14 - protein, 12 aa, Artificial Sequence
  Description of Artificial Sequenceamino- terminal extracellular domain peptide of  histamine type 2 receptor (H2R)
SCCLDSIALKVT

(19) United States
(12) Patent Application Publication
(10) Pub. No.: US 2009/0317357 A1
(43) Pub. Date: Dec. 24, 2009

(54) Methods for treating or preventing autoimmune disease using histamine h1 receptor-blocking agents

(76) Inventors: Lawrence Steinman, Stanford, CA (US); Rosetta Pedotti, Milan (IT)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/546,569

(22) PCT Filed: Feb. 24, 2004
(86) PCT No.: PCT/US04/05359

§ 371 Date: Aug. 22, 2005

(30) Foreign Application Priority Data
Feb. 24, 2003 (US) 60450118

(51) Int. Cl. A61K 38/20; A61K 39/00; A61K 31/7088; A61K 31/132

(52) U.S. Cl. 424/85.2; 424/185.1; 514/44 R; 514/673

(57) ABSTRACT

Methods for treating or preventing an autoimmune disease using agents that block the histamine H1 receptor are disclosed. H1 receptor-blocking agents useful in accordance with the methods provided herein include, for example, H1 antihistamines, particularly H1 antihistamines that do not substantially block the serotonin receptor.

### CROSS-REFERENCES TO RELATED APPLICATIONS

This application claims benefit of U.S. Provisional Application No. 60/450,118, filed Feb. 24, 2003, which is incorporated by reference herein in its entirety.

### STATEMENT AS TO RIGHTS TO INVENTIONS MADE UNDER FEDERALLY SPONSORED RESEARCH AND DEVELOPMENT

This work was supported by a grant from the National Institutes of Health. The U.S. government may have certain rights in the invention.

### BACKGROUND OF THE INVENTION

Autoimmunity and anaphylactic allergy have classically been considered as dichotomous disease conditions driven by different immune response pathways. Autoimmune-type responses are initiated primarily by antigen-specific T or B lymphocytes. T cell-mediated responses have been shown to predominantly include proliferation and infiltration of CD4⁺ Th1 cells, CD8⁺ cytotoxic T cells, and macrophages in target tissues, whereas B cell-mediated autoimmune disorders, in addition to B lymphocyte involvement, are predominated by complement and antibodies of the IgG or IgM class. Further, autoimmune responses are reminiscent of type II (cytolytic), type III (immune complex), and/or type IV (delayed type) hypersensitivity reactions, which typically have onsets occurring hours or days after challenge with antigen. In contrast, anaphylactic responses, which characterize type I hypersensitivity allergic reactions and which typically have a rapid onset within minutes of antigenic challenge, are primarily mediated by IgE antibodies, which bind to Fc receptors on mast cells and basophils. Cross-linking of IgE antibodies by antigen triggers the mast cells and basophils to release pharmacologically active agents responsible for the anaphylaxis. This dichotomy between autoimmunity and anaphylactic allergy is further underscored for T cell-mediated autoimmune responses by the Th1/Th2 paradigm, with Th2 cytokines (e.g., IL4, which controls the switch to IgE synthesis) predominating in allergy, in contrast to the preponderance of Th1 pathways in T cell-mediated autoimmune responses.

Some recent studies have focused on elements of allergic responses in the development of autoimmune disorders. For example, it is possible to induce “horror autotoxicus” with anaphylaxis against certain self antigens. (Pedotti et al., Nat. Immunol. 2:216-22, 2001.) In addition, mast cells and other elements that can participate in allergic responses are present in multiple sclerosis (MS) lesions (see Olsson, Acta Neurol. Scand. 50:611-618, 1974; Toms et al., J. Neuroimmunol 30:169-177, 1990; Brenner et al., J. Neurol. Sci. 122:210-213, 1994; Ibrahim et al., J. Neuroimmunol. 70:131-138, 1996), and platelet activating factor (PAF) and mast cell tryptase are elevated in the spinal fluid during MS relapses (Callea et al., J. Neuroimmunol 94:212-221, 1999; Rozniecki et al., Ann. Neurol. 37:63-66 (1995)). Further, in mice, antagonists of the receptor for serotonin, a mast cell mediator, can ameliorate experimental autoimmune encephalomyelitis (EAE), a model for MS (Dietsch & Hinrichs, J. Immunol 142:1476-81, 1989; Linthicum, Immnuobiology 162:211-20, 1982), and blockade of mast cell biogenic amine secretion has also been shown to reduce the severity and progression of EAE (Dimitriadou et al., Intl. J. Immunopharmacol. 22:673-684, 2000).

In contrast, studies to date have not shown a role for histamine, a major preformed allergic mediator, in the development of autoimmunity. Histamine, formed by the decarboxylation of the amino acid histidine, is stored in mast cells and basophil secretory granules. When released, histamine binds rapidly to a variety of cells via different histamine receptor subtypes, including H1 histamine receptors (H1R), which mediate the response antagonized by conventional antihistamines. Previous investigators have suggested that amelioration of autoimmune disease symptoms with non-selective “antihistaminic” agents having anti-serotonin or neurogenic mast cell secretion inhibitory activity is not attributable to blockade of H1R pathways. (See Dietsch & Hinrichs; Dimitriadou et al.) Further, reduction of EAE symptoms or progression through a H1R blockade mechanism has not been shown using H1R-selective agents. (See Linthicum; Dimitriadou et al.)

Current approaches for treating autoimmune disorders, which target those immune response pathways classically implicated in the development of autoimmunity, are only partially effective in ameliorating disease. Such therapies include interferon β, glatiramer acetate, high dose IV immunoglobulin (IVIg), steroids, methotrexate, and cyclophosphamide. (See, e.g., Hanson & Cafruny, S. D. J. Med. 55:477-81, 2002; Comi & Moiola, Neuroglia 17:244-58, 2002) Further, the use of these available immunomodulatory agents for autoimmune disease is often limited by route of administration, cost, or dose-limiting side effects, particularly those resulting from the actions of the agent on non-target tissues.

Therefore, there is a need in the art for new methods of autoimmune disease treatment that target different compartments of the immune response. Methods that target other pathways can offer advantageous alternative or conjunctive approaches to these current treatments. The present invention meets these and other needs.

### BRIEF SUMMARY OF THE INVENTION

The present invention provides methods for treating or preventing an autoimmune disease in a subject by administering to the subject an effective amount of an agent that blocks histamine H1 receptor (H1R), wherein the agent excludes cyproheptadine or hydroxyzine.

In certain embodiments, the H1R-blocking agent is an H1-antihistamine. The H1-antihistamine can be, for example, an alkylamine, an ethanolamine, an ethylenediamine, a phenothiazine, a piperidine, or a piperazine. In addition, the H1-antihistamine can be, for example, a first-generation antihistamine. Also, the H1-antihistamine can lack a carboxylate moiety. In one embodiment, the H1-antihistamine is pyrilamine.

In yet other variations, the H1R-blocking agent does not substantially block serotonin receptor or mast cell biogenic amine secretion. For example, in specific embodiments, the ED50 dose for inhibition of the serotonin receptor by the agent is at least about 0.5 mg/kg, at least about 0.6 mg/kg, or at least about 0.8 mg/kg.

The autoimmune disease treated or prevented according to the methods of the invention can be, for example, rheumatoid arthritis, graft-versus host disease (GvHD), inflammatory bowel disease (IBD), insulin dependent diabetes mellitus (IDDM), multiple sclerosis, primary biliary cirrhosis, systemic sclerosis, psoriasis, autoimmune thyroiditis, or autoimmune thrombocytopenic purpura. In certain embodiments, the autoimmune disease treated or prevented is a Th1-mediated autoimmune disease. The Th-1 mediated autoimmune disease can be, for example, an autoimmune demyelinating disease. In one embodiment, the Th1-mediated autoimmune disease is an autoimmune demyelinating disease. In yet other embodiments, the autoimmune demyelinating disease is multiple sclerosis. Further, the autoimmune disease can be, for example, a relapsing-remitting form of the disease; in these embodiments, the administration of the agent can, for example, decrease the relapse rate of the disease.

Subjects treated are typically diagnosed with an autoimmune disease. Subjects can optionally be monitored for a change in a symptom of the autoimmune disease in response to the treatment. In certain embodiments, the subject does not have a second disease or disorder that requires treatment with the H1R-blocking agent.

The H1R-blocking agents can be administered, for example, by intramuscular, subcutaneous, intravenous, parenteral, intranasal, intrapulmonary, or oral routes of administration.

In certain embodiments, the H1R-blocking agent is not co-administered with a second active agent that is a dithiocarbamate disulfide derivative; substituted 1,4-dihydropyridine bradykinin antagonist; heteroaryl substituted 1,4-dihydropyridine bradykinin antagonist; LTB-receptor antagonist comprising a disubstituted phenyl-benzamidine derivative; or a small molecule antagonist of chemokine receptor CCR1.

In yet other embodiments, the H1R-blocking agent is co-administered with a second active agent. The second active agent can be, for example, a self-vector that includes a polynucleotide encoding a self-polypeptide associated with the autoimmune disease for which treatment or prevention is sought; an immunomodulatory protein; or a vector encoding an immunomodulatory protein. In certain embodiments, the self-vector and the vector encoding an immunomodulatory protein are co-administered.

Immunomodulatory proteins suitable for use according to the methods of the present invention include, for example, cytokines or chemokines. In certain embodiments, the immunomodulatory protein is a cytokine that is IL-4, IL-10, or IL-13.

Further, where the second active agent is a self-vector, an immune modulatory sequence can optionally be co-administered to the subject. The immune modulatory sequence can be, for example, 5′-Purine-Pyrimidine-[X]-[Y]-Pyrimidine-Pyrimidine-3′ or 5′-Purine-Purine-[X]-[Y]-Pyrimidine-Pyrimidine-3′, wherein X and Y are any naturally occurring or synthetic nucleotide, except that X and Y cannot be cytosine-guanine.

In addition, in embodiments where the second active agent is a self-vector and the autoimmune disease is multiple sclerosis, the self-polypeptide can be, for example, myelin basic protein (MBP), proteolipid protein (PLP), myelin associated glycoprotein (MAG), cyclic nucleotide phosphodiesterase (CNPase), myelin-associated oligodendrocytic basic protein (MBOP), myelin oligodendrocyte protein (MOG), or alpha-B crystalline. Where the autoimmune disease is insulin dependent diabetes mellitus, the self-polypeptide can be, for example, insulin, insulin B chain, preproinsulin, proinsulin, glutamic acid decarboxylase 65 kDa and 67 kDa forms, tyrosine phosphatase IA2 or IA-2b, carboxypeptidase H, heat shock proteins, glima 38, islet cell antigen 69 kDa, p52, or islet cell glucose transporter (GLUT 2).

### BRIEF DESCRIPTION OF THE DRAWINGS

FIGS. 1(A & B). Allergy-related gene expression in CNS of mice with EAE and in Th1 and Th2 T cell lines activated against a myelin peptide. (A) EAE was induced with PLP p 139-151 in SJL mice; brain (a, c, d) and spinal cord (b, d, f) were removed at different time points of the disease and analyzed by quantitative PCR. The relative expression of PAFR (a, b), PGDS (c, d), and MMCP-7 (e, f) was quantified using primers specific for the target (see Example 2) and normalization against β3-Actin. Means of qPCR values of 3 to 5 animals±standard deviation per time point are represented. (B) The relative expression of histamine receptors 1 and 2 was quantified in a Th1 and Th2 type T cell line specific for PLP139-151. Data are representative of 2 consecutive experiments. P=0.009 for H1R (Th1 versus Th2) by ANOVA; P=0.004 for H2R (Th1 versus Th2) by ANOVA.

FIGS. 2(A-D). Expression of H1R and H2R in the CNS of SJL mice with EAE induced with PLP 139-151. Brains were obtained 20 days after disease induction and cryostat sections were stained with rabbit polyclonal antibodies against H1R and H2R. H1R (A) and H2R (B) are expressed on mononuclear cells (arrowheads) in perivascular inflammatory foci. Parenchymal cells consistent with microglia, astrocytes, and infiltrating inflammatory cells (arrows) are also stained. In brains of naïve SJL mice, H1R (C) is not detected, although rare astrocytes and choroid plexus cells were stained (not shown). H2R (D) is expressed on microvascular endothelial cells (arrows). Original magnifications: A, C, 240×; B, D, 320×.

FIGS. 3(a-c). Amelioration of EAE in FcγRIII deficient mice. EAE was induced in FcγRIII−/−(n=12) and +/+(n=12) with MOG 35-55. (a) FcγRIII−/−mice have a significantly milder disease compared to FcγRIII+/+mice (data are shown as mean±SEM) and (b) they are protected from EAE related death (0 of 12 in the FcγRIII−/− mice vs 4 of 12 in the FcγRIII+/+). (c) EAE is more remitting in FcγRIII−/− mice, with 56% (5 of 9) presenting periods of complete remissions compared to 17% (2 of 12) of the wild type mice.

FIG. 4. Modulation of EAE with H1R antagonist and PAFR antagonist. EAE was induced in SJL/J mice with PLP139-151. The H1R antagonist pyrilamine (0.6 mg/mouse; n=8), PAFR antagonist CV6209 (1 μg/mouse; n=8), or vehicle alone (PBS; n=7) were given daily i.p. starting on day 2 after the induction of EAE.

### DETAILED DESCRIPTION OF THE INVENTION

The present invention provides methods for treating or preventing autoimmune disease in a subject by administering an effective amount of agent that blocks the H1 receptor for histamine (H1R). The invention relates to applicants' surprising discovery that blocking H1R, a receptor involved in the anaphylactic allergic response, ameliorates the manifestation of clinical disease symptoms of autoimmune disease. The methods provided herein offer an advantageous alternative or conjunctive approach for controlling the course of autoimmune diseases, including reducing the relapse rate or severity of a relapse in relapsing-remitting disease.

In certain preferred embodiments, the agent is an antihistamine drug as defined herein. Antihistamine drugs traditionally have oral routes of administration, which are less invasive than many currently approved drugs for autoimmune disease. In addition, the collateral effects of antihistamine drugs are fewer and less severe compared to current therapeutic approaches. Also, such drugs are well-known in the medical practice for other disease conditions, including allergy and asthma. Because many antihistamines are already approved by the FDA for other disease conditions, their efficacy can be tested directly in phase II or III clinical trials, thereby reducing development costs. Further, the costs related to the production and storage of antihistamines are significantly lower than many currently approved drugs.

Prior to setting forth the invention in more detail, it may be helpful to a further understanding thereof to set forth definitions of certain terms as used hereinafter.

### DEFINITIONS

Unless defined otherwise, all technical and scientific terms used herein have the same meaning as commonly understood by one of ordinary skill in the art to which this invention belongs. Although any methods and materials similar to those described herein can be used in the practice or testing of the present invention, only exemplary methods and materials are described. For purposes of the present invention, the following terms are defined below.

The terms “a,” “an,” and “the” as used herein include plural referents unless the context clearly dictates otherwise.

The terms “molecule,” “compound,” and “agent” as used herein are synonymous and are used broadly to mean molecules that are potentially capable of structurally interacting with proteins through non-covalent interactions, such as, for example, through hydrogen bonds, ionic bonds, van der Waals attractions, or hydrophobic interactions. For example, agents most typically include functional groups necessary for structural interaction with proteins, particularly those groups involved in hydrogen bonding. Agents can include, for example, a small molecule drug; a peptide, including a variant analog, homolog, modified peptide or peptide-like substance such as a peptidomimetic or peptoid; or a protein such as an antibody or a fragment thereof, such as an Fᵥ, F_{c}, or F_{ab} fragment of an antibody, which contains a binding domain. An agent can be nonnaturally occurring, produced as a result of in vitro methods, or can be naturally occurring, such as, for example, a protein or fragment thereof expressed endogenously in a cell or from a cDNA library.

The term “polypeptide” refers to a polymer of amino acids and its equivalent and does not refer to a specific length of the product; thus, peptides, oligopeptides and proteins are included within the definition of a polypeptide. A “fragment” refers to a portion of a polypeptide typically having at least 10 contiguous amino acids, more typically at least 20, still more typically at least 50 contiguous amino acids of the polypeptide. A derivative is a polypeptide having conservative amino acid substitutions, as compared with another sequence. Derivatives further include, for example, glycosylations, acetylations, phosphorylations, and the like. Further included within the definition of “polypeptide” are, for example, polypeptides containing one or more analogs of an amino acid (e.g., unnatural or “non-classical” amino acids, and the like), polypeptides with substituted linkages as well as other modifications known in the art, both naturally and non-naturally occurring.

The terms “polynucleotide” and “nucleic acid” are used synonymously and refer to a polymer composed of a multiplicity of nucleotide units (ribonucleotide or deoxyribonucleotide or related structural variants) linked via phosphodiester bonds. Polynucleotides and nucleic acids include RNA, DNA, synthetic forms, and mixed polymers, both sense and antisense strands, and can also be chemically or biochemically modified or can contain non-natural or derivatized nucleotide bases.

The term “receptor” means a molecule, present on the extracellular surface of a cell, that is specialized to detect changes in the cell's environment and trigger various actions. Receptors act as a switch through the binding and unbinding of molecules.

The term “agonist” as used herein means a molecule that binds to a receptor and upregulates its function as a result of the binding. Agonists can function, for example, by inducing a conformational change of the receptor to an active state; or by stabilizing, upon binding, a naturally occurring active conformation (e.g. where active and inactive conformations exist in equilibrium), thereby shifting the equilibrium to an active state. Agonists can trigger a cascade of molecular binding and/or enzymatic biochemical events within (e.g., intracellular cell signaling) or outside (e.g., complement cascade) of the cell on which the receptor resides.

The term “antagonist” as used herein refers to a molecule or agent that binds to a receptor and downregulates its function as a result of the binding. Antagonists can function, for example, as conventionally understood in the art, by preventing the binding of agonist molecules via direct competition, thereby blocking the biological actions of the agonist. In addition, as used herein, “antagonist” also refers to molecules that can act as “inverse agonists,” i.e., by binding to and stabilizing the inactive conformation of a receptor that naturally exists in equilibrium between active and inactive states, thereby shifting the equilibrium towards the inactive state.

The term “antihistamine” as used herein refers to a class of small organic pharmacologic agents that act as histamine receptor antagonists. As used herein, antihistamines refers to antagonists of H1, H2, H3, or H4 receptors. “H1-antihistamines,” i.e., antihistamines that block H1 receptors, are well-known in the art (see, e.g., Passalacqua et al., in Histamine and H1-Antihistamines in Allergic Disease 65-100 (F. Estelle R. Simons ed., 2002), incorporated by reference herein) and include, for example, the first-generation, centrally acting H1 receptor antagonists (e.g., diphenhydramine) and the new, second-generation nonsedating H1 blockers (e.g., loratadine). H1-blocking antihistamines include well-known structural classes such as alkylamines, ethanolamines, ethylenediamines, phenothiazines, piperidines, and piperazines. Polypeptide agents (e.g., peptides, antibodies) are excluded from the definition of antihistamines as used herein.

“H1R-blocking antibody” and “H1R-blocking peptide” refer to antibody (or fragment thereof) or peptide, respectively, that acts as an H1 receptor antagonist.

The term “autoimmune disease” refers to any disorder having a pathogenesis characterized at least in part by adaptive immunity that becomes misdirected at healthy cells and/or tissues of the body. Autoimmune diseases are characterized by T and/or B lymphocytes that aberrantly target self-molecules (e.g., self-polypeptides), causing injury and/or malfunction of an organ, tissue, or cell-type within the body (e.g., pancrease, brain, thyroid, or gastrointestinal tract). Autoimmune diseases include disorders that affect specific tissues as well as multiple tissues. Further, “autoimmune disease” as used herein can include acute, chronic, and/or relapsing-remitting forms of a disease. Examples of autoimmune diseases include rheumatoid arthritis, graft-versus host disease (GvHD), inflammatory bowel disease (IBD), insulin dependent diabetes mellitus (IDDM), multiple sclerosis, primary biliary cirrhosis, systemic sclerosis, psoriasis, autoimmune thyroiditis, and autoimmune thrombocytopenic purpura.

The term “Th1-mediated autoimmune disease” refers to an autoimmune disease that is T cell-mediated and characterized by a primarily Th1 cytokine profile. While Th1 cytokines predominate, “Th1-mediated autoimmune disease” is not mutually exclusive with, and therefore can include, autoimmune pathology also characterized by other immune response pathways such as, e.g., Th2- or Th0-type responses.

The term “inhibit” or “block” as used herein means to reduce (e.g., immune response, receptor activation, autoimmune disease symptom, etc.) by a measurable amount, or to prevent entirely.

“Treating,” “treatment,” or “therapy” of a disease or disorder means slowing, stopping or reversing the disease's progression, as evidenced by a reduction or elimination of either clinical or diagnostic symptoms, using the methods of the present invention as described herein. Treatment can include a decrease in the severity of symptoms in acute or chronic disease as well as a decrease in the relapse or exacerbation rate in relapsing-remitting disease. In the preferred embodiment, treating a disease means reversing or stopping the disease's progression. As used herein, ameliorating a disease and treating a disease are equivalent.

“Preventing,” “prophylaxis,” or “prevention” of a disease or disorder means prevention of the occurrence or onset of a disease or disorder or some or all of the its symptoms.

The terms “subject” herein means any mammalian patient to which the H1R-blocking agents may be administered according to the methods of the present invention. Subjects specifically intended for treatment using the methods described herein include humans.

The term “effective amount” in context of administration of an agent, refers to an amount of a molecule that is sufficient to modulate an autoimmune response in a subject so as to inhibit the occurrence or ameliorate one or more symptoms of the target autoimmune response in the subject. An effective amount of an agent is administered according to the methods of the present invention in an “effective regime.” The term “effective regime” refers to a combination of amount of the agent and dosage frequency adequate to accomplish treatment or prevention of the autoimmune disease.

“Self-polypeptide” as used herein refers to any polypeptide, or fragment or derivative thereof, that is encoded within the genome of the animal, is expressed in the animal, may be modified posttranslationally at some time during the life of the animal, and is associated with an autoimmune disorder as a self-antigen. Examples of posttranslational modifications of self-polypeptides are glycosylation, addition of lipid groups, dephosphorylation by phosphatases, addition of dimethylarginine residues, citrullination of fillagrin and fibrin by peptidyl arginine deiminase (PAD); alpha B crystallin phosphorylation; citrullination of MBP; and SLE autoantigen proteolysis by caspases and granzymes. “Antigen” refers to any molecule that can be specifically recognized by components of the immune response such as lymphocytes or antibodies. Self-polypeptide does not include immune proteins which are molecules expressed specifically and exclusively by cells of the immune system for the purpose of regulating immune function. Certain immune proteins that are included in the definition of self-polypeptide and they are: class I MHC membrane glycoproteins, class II MHC glycoproteins, and osteopontin.

“Self-vector” means a vector which comprises a polynucleotide encoding one or more self-polypeptides. Self-vectors encompassed by the present invention are further defined in U.S. patent application Ser. No. 10/302,098, incorporated by reference herein in its entirety.

“Modulation of an immune response” as used herein refers to any alteration of an existing or potential immune response in vitro or in vivo. In the context of autoimmune disease, such alteration is of an immune response against self-molecules. Modulation can include any alteration in the presence or function of any immune cell (e.g., T cell, B cell, NK cell, macrophage, dendritic cell, neutrophil, mast cell, basophil, and the like) involved in or having the potential to be involved in the immune response. Modulation includes, for example, alteration in the expression and/or function of genes, proteins and/or other molecules in immune cells as part of an immune response; elimination, deletion, or sequestration of immune cells; induction or generation of immune cells that can modulate the functional capacity of other cells such as, e.g., autoreactive lymphocytes, antigen presenting cells (APCs), or inflammatory cells; induction of an unresponsive state in immune cells (e.g., anergy); or increasing, decreasing, or changing the activity or function of immune cells. Alteration in the pattern of proteins expressed by immune cells can include, for example, altered production and/or secretion of certain classes of molecules such as cytokines (e.g., IL-2, IFN-γ, TNF-α, IL-4), chemokines, growth factors, transcription factors (e.g., NF-κB), kinases (e.g. Lck, Lyn), phosphatases (e.g., PTP-1C, PTP-1D), costimulatory molecules (e.g., B7.1/B7.2, CTLA-4, CD40, ICAM, LFA-1), or other cell surface receptors.

“Immune Modulatory Sequences (IMSs)” as used herein refers to compounds consisting of deoxynucleotides, ribonucleotides, or analogs thereof that modulate an autoimmune or inflammatory disease. IMSs may be oligonucleotides or a sequence of nucleotides incorporated in a vector. IMSs for use according to the methods provided herein are further described in U.S. patent application Ser. No. 10/302,098.

“Immunomodulatory protein” as used herein refers to a polypeptide molecule (e.g., protein, glycoprotein, peptide, and the like), known to modulate a host's immune response. Immunomodulatory proteins can include recombinant forms of the protein. Immunomodulatory proteins include, for example, cytokines (or functional fragments thereof) such as, e.g., interleukins, interferons, or colony stimulating factors. Immunomodulatory proteins can also include, for example, chemokines or costimulatory molecules or functional fragments thereof. Where the native protein is a membrane bound molecule (e.g., receptors such as cytokine receptors (e.g., TNF-α R, IL-2R) or costimulatory molecules such as, for example, CD40, CTLA-4, or B7 molecules), the immunomodulatory protein as used in the methods described herein can be a soluble form of the protein, such as, for example, an Ig fusion protein. Methods for making soluble Ig fusion recombinant forms of receptors are known in the art (see, e.g., U.S. Pat. No. 5,750,375).

The term “active agent” means any agent that can modulate an immune response.

“Dithiocarbamate disulfide derivatives” refers to disulfide derivatives of dithiocarbamates having structure (I) as defined and disclosed in U.S. Pat. No. 6,093,743, incorporated by reference herein.

“Substituted 1,4-dihydropyridine bradykinin antagonists” refers to compounds having formula (I) as defined and disclosed in US Patent Application Publication No. 2002/0042421 A1, incorporated by reference herein.

“Heteroaryl substituted 1,4-dihydropyridine bradykinin antagonists” refers to compounds having formula (I) as defined and disclosed in US Patent Application Publication No. 2001/0046993 A1, incorporated by reference herein.

“LTB-receptor antagonists comprising disubstituted phenyl-benzamidine derivatives” refers to compounds having formula (I) as defined and disclosed in U.S. Pat. No. 6,291,531, incorporated by reference herein.

“Small molecule antagonists of chemokine receptor CCR1” refers to compounds having formulas (I), (Ia), (II), (III), (IV), (IVa), (IVb), (V), (VI), (VI), (VIIa)-(VIIk), (VIII), (IX), (X), and (XI) as defined and disclosed in International Publication No. WO 01/09138 A2, incorporated by reference herein.

“Substantially block serotonin receptor” as used herein refers to a characteristic of the agent as determined by an independent in vivo animal model standard. “Substantially block serotonin receptor” means that the ED₅₀ dose for inhibition of the serotonin receptor by the agent, as determined by the methods described in Stone et al., J. Pharmocol. Exptl. Therap. 131:73-84, 1961, incorporated by reference herein, is at least about 0.1 mg/kg, typically at least about 0.2 mg/kg, more typically at least about 0.5 mg/kg, preferably at least about 0.6 mg/kg, more preferably at least about 0.7 mg/kg, and even more preferably at least about 0.8 mg/kg, or at least about 1.0 mg/kg, or at least about 2.0 mg/kg i.v.

“Substantially block biogenic amine secretion” in the context of H1R-blocking agents as used herein, refers to a characteristic of the agent as determined by an independent in vivo animal model standard. “Substantially block biogenic amine secretion” means that inhibition of mast cell degranulation in an area of autoimmune disease involvement, following administration of the agent at a dose of 2.0 to 2.5 mg/kg in an animal model for the autoimmune disease, is no more than about 40%, typically no more than about 25%, more typically no more than about 15%, and most typically no more than about 5%. Such inhibition is determined by comparing the extent of degranulation of mast cells in animals treated with the agent with the extent of such degranulation of mast cells in animals not exposed to the agent. Degranulation is determined by evaluation of staining with toluidine blue using criteria set forth in Dimitriadou et al., Intl. J. Immunopharmacol. 22:673-684, 2000, incorporated by reference herein.

### Autoimmune Diseases

The present invention provides methods for treating or preventing autoimmune disease. Progression of disease can be measured by monitoring clinical or diagnostic symptoms using known methods such as, for example, methods described infra. The methods according to the present invention are amenable to the treatment or prevention of autoimmune disorders characterized at least in part by anaphylactic allergic responses to self. As shown by the present inventors herein, multiple elements of allergic responses are involved in the modulation of autoimmune disease. The methods described herein provide a means for inhibiting these allergic immune responses to self (i.e., through H1R blockade), thereby reducing the course and/or severity of the autoimmune response. Blockade of the H1 receptor can, for example, decrease vascular permeability, thereby inhibiting the infiltration of immune cells to target tissues. For example, permeabilization of the blood-brain barrier is necessary for the entry in the central nervous system of the immune cells causing myelin damage in autoimmune demyelinating diseases such as multiple sclerosis and EAE (experimental autoimmune encephalomyelitis). Further, H1R blockade may act by reducing the proinflammatory activity of immune cell infiltrates at target sites, including antigen-specific Th1 cells preferentially expressing the H1 receptor. In certain embodiments, the autoimmune disease is a Th1-mediated autoimmune disease. In one exemplary embodiment, the autoimmune disease is an autoimmune demyelinating disease (e.g., multiple sclerosis or EAE). In yet other embodiments, the autoimmune disease is a relapsing-remitting form of the disease; in certain embodiments for treatment of relapsing-remitting disease, the treatment according to the methods provided herein decrease the relapse rate of the disease.

Several examples of autoimmune diseases that can be treated according to the methods provided herein are described below.

Multiple Sclerosis: Multiple sclerosis (MS) is the most common demyelinating disorder of the CNS. Onset of symptoms typically occurs between 20 and 40 years of age and manifests as an acute or sub-acute attack of unilateral visual impairment, muscle weakness, paresthesias, ataxia, vertigo, urinary incontinence, dysarthria, or mental disturbance (in order of decreasing frequency). Such symptoms result from focal lesions of demyelination which cause both negative conduction abnormalities due to slowed axonal conduction, and positive conduction abnormalities due to ectopic impulse generation (e.g., Lhermitte's symptom). Diagnosis of MS is based upon a history including at least two distinct attacks of neurologic dysfunction that are separated in time, produce objective clinical evidence of neurologic dysfunction, and involve separate areas of the CNS white matter. Laboratory studies providing additional objective evidence supporting the diagnosis of MS include magnetic resonance imaging (MRI) of CNS white matter lesions, cerebral spinal fluid (CSF) oligoclonal banding of IgG, and abnormal evoked responses. Although most patients experience a gradually progressive relapsing remitting disease course, the clinical course of MS varies greatly between individuals and can range from being limited to several mild attacks over a lifetime to fulminant chronic progressive disease. Accordingly, several subtypes or stages of MS are known and include benign MS, relapsing-remitting MS, secondary-progressive MS, primary-progressive MS, and progressive-relapsing MS. A quantitative increase in myelin-autoreactive T cells with the capacity to secrete IFN-γ is associated with the pathogenesis of MS and EAE.

Rheumatoid Arthritis: Rheumatoid arthritis (RA) is a chronic autoimmune inflammatory synovitis that causes erosive joint destruction. RA is mediated by T cells, B cells, and macrophages. Evidence that T cells play a critical role in RA includes the (1) predominance of CD4+ T cells infiltrating the synovium, (2) clinical improvement associated with suppression of T cell function with drugs such as cyclosporine, and (3) the association of RA with certain HLA-DR alleles. The HLA-DR alleles associated with RA contain a similar sequence of amino acids at positions 67-74 in the third hypervariable region of the β chain that are involved in peptide binding and presentation to T cells. RA is mediated by autoreactive T cells that recognize a self-protein, or modified self-protein, present in synovial joints.

Insulin Dependent Diabetes Mellitus: Human type I or insulin-dependent diabetes mellitus (IDDM) is characterized by autoimmune destruction of the β cells in the pancreatic islets of Langerhans. The depletion of β cells results in an inability to regulate levels of glucose in the blood. Overt diabetes occurs when the level of glucose in the blood rises above a specific level, usually about 250 mg/dl. In humans a long presymptomatic period precedes the onset of diabetes. During this period there is a gradual loss of pancreatic beta cell function. The development of disease is implicated by the presence of autoantibodies against insulin, glutamic acid decarboxylase, and the tyrosine phosphatase IA2 (IA2). Markers that may be evaluated during the presymptomatic stage are the presence of insulitis in the pancreas, the level and frequency of islet cell antibodies, islet cell surface antibodies, aberrant expression of Class II MHC molecules on pancreatic beta cells, glucose concentration in the blood, and the plasma concentration of insulin. An increase in the number of T lymphocytes in the pancreas, islet cell antibodies and blood glucose is indicative of the disease, as is a decrease in insulin concentration.

Autoimmune Uveitis: Autoimmune uveitis is an autoimmune disease of the eye. Autoimmune uveitis is currently treated with steroids, immunosuppressive agents such as methotrexate and cyclosporin, intravenous immunoglobulin, and TNFα-antagonists.

Experimental autoimmune uveitis (EAU) is a T cell-mediated autoimmune disease that targets neural retina, uvea, and related tissues in the eye. EAU shares many clinical and immunological features with human autoimmune uveitis, and is induced by peripheral administration of uveitogenic peptide emulsified in Complete Freund's Adjuvant (CFA).

Primary Billiary Cirrhosis: Primary Biliary Cirrhosis (PBC) is an organ-specific autoimmune disease characterized by progressive destruction of intrahepatic biliary epithelial cells (IBEC) lining the small intrahepatic bile ducts. This leads to obstruction and interference with bile secretion, causing eventual cirrhosis. Association with other autoimmune diseases characterized by epithelium lining/secretory system damage has been reported, including Sjögren's Syndrome, CREST Syndrome, Autoimmune Thyroid Disease and Rheumatoid Arthritis. Attention regarding the driving antigen(s) has focused on the mitochondria for over 50 years, leading to the discovery of the antimitochondrial antibody (AMA) (Gershwin et al., Immunol. Rev. 174:210-225, 2000); (Mackay et al., Immunol. Rev. 174:226-237, 2000). AMA soon became a cornerstone for laboratory diagnosis of PBC, present in serum of 90-95% patients long before clinical symptoms appear. Studies identifying the role of pyruvate dehydrogenase complex (PDC) antigens in the etiopathogenesis of PBC support the concept that PDC plays a central role in the induction of the disease (Gershwin et al.; Mackay et al.). PBC is treated with glucocorticoids and immunosuppressive agents including methotrexate and cyclosporin A.

A murine model of experimental autoimmune cholangitis (EAC) uses intraperitoneal (i.p.) sensitization with mammalian PDC in female SJL/J mice, inducing non-suppurative destructive cholangitis (NSDC) and production of AMA (Jones, J. Clin. Pathol. 53:813-21, 2000).

Other autoimmune diseases that can be treated according to the methods provided herein include, for example, graft-versus host disease (GvHD), inflammatory bowel disease (IBD), systemic sclerosis, psoriasis, autoimmune thyroiditis, and autoimmune thrombocytopenic purpura.

Agents that Block Histamine H1 Receptor (H1R)

The methods according to the present invention for treating or preventing autoimmune disease comprise the use of agents that block activation of the H1 receptor for histamine. H1R-blocking agents can include, for example, antagonists of H1R (e.g., agents that bind to the receptor and thereby prevent the receptor's binding of histamine or, alternatively, “inverse agonists,” as described supra, that stabilize the inactive conformation of the H1 receptor). Such antagonists can be competitive inhibitors, binding to the same site as histamine, or non-competitive inhibitors, binding to an allosteric site of the receptor. Further, the agent can act as an inhibitor of H1R activation intracellularly by, for example, binding to a G-protein binding site within the cytoplasmic domain of the receptor to inhibit the formation of [H1 receptor-G-protein-GDP] intermediates. H1R blocking agents can also include soluble agents that bind directly to histamine with sufficient affinity to outcompete histamine receptors, thereby preventing binding of histamine to the H1 receptor on the cell surface (e.g., “high affinity histamine binding proteins” (HBPs) such as, for example, described in Paesen et al., Mol. Cell 3:661-71, 1999). In certain embodiments of the invention, the antihistamine does not substantially block serotonin receptor or biogenic amine secretion.

Antihistamines: In one embodiment of the invention, the H1R-blocking agent is an antihistamine drug. Antihistamines that block the H1 receptor, including their classification and structure, are known in the art. (See, e.g., Passalacqua et al., supra. See also Zhang et al., in Burger's Medicinal Chemistry and Drug Discovery: Therapeutic Agents (Wolff, M. E., ed., 1997) vol. 5, 5th Ed., pp. 495-559, John Wiley & Sons, Inc., New York, (incorporated by reference herein).) Typically, H1 antihistamines are inverse agonists as defined supra, down-regulating constitutive receptor activity by binding and stabilizing the H1 receptor in its inactive state (see, e.g., Leurs et al., Clin. Exp. Allergy 32:489-98, 2002). H1-antihistamines encompassed within the methods described herein include, for example, brompheniramine, triprolidine, clemastine, diphenhydramine, bromodiphenhydramine, doxylamine, tripelennamine, pyrilamine, promethazine, fexofenadine, loratadine, cetrizine, meclizine, pheniramine, chlorpheniramine, brompheniramine, dexbrompheniramine, dexchlorpheniramine, dimenhydrinate, tripelenamine, phenyltoloxamine, terfenadine, acrivastine, doxylamine, phenindamine, epinastine, mequitazine, mianserine, ebastine, mizolastine, levocabastine, astemizole, antazoline, methapyriline, carbinoxamine, dimethindene, methdilazine, trimeprazine, cyclizine, buclizine, chlorcyclizine, azelastine, ketotifen, and oxatomide. H1-antihistamines fall within known structural classes that include alkylamines (e.g., brompheniramine, triprolidine), ethanolamines (e.g., clemastine, diphenhydramine, doxylamine), ethylenediamines (e.g., tripelennamine, pyrilamine), phenothiazines (e.g., promethazine), piperidines (e.g., fexofenadine, loratadine), and piperazines (e.g., cetrizine, meclizine). Also, antihistamines can be classified in clinical terms as, for example, “first generation,” potentially sedating H1-antihistamines (e.g., chlorpeniramine, diphenhydramine, promethazine, and triprolidane) or “second generation,” relatively non-sedating H1-antihistamines (e.g., cetrizine, ebastine, fexofenadine, loratadine, and mizolastine).

Also, H1-antihistamines are further characterized by a known three-dimensional pharmacophoric model, which includes cis- and trans-aromatic rings positioned relative to the C_{α} and C_{β} carbon atoms of H1R Asp¹¹⁶, which is involved in the binding of the protonated amine function found in both agonists and antagonists structures. (See, e.g., Wieland et al., J. Biol. Chem. 274:29994-30000, 1999, incorporated by reference herein.) Further, the carboxylate moiety of second generation H1-antihistamines is believed to act as a specific anchor point for these antihistamines through an interaction with H1R Lys²⁰⁰. (See Wieland et al.)

In certain embodiments of the invention, the H1-antihistamine is an alkylamine, an ethanolamine, an ethylenediamine, a phenothiazine, a piperidine, or a piperazine. In other embodiments of the invention, the H1-antihistamine is a first generation H1-antihistamine. In yet other embodiments, the H1-antihistamine is a second generation H1-antihistamine. Further, in another embodiment, the H1-antihistamine is an H1-antihistamine lacking a carboxylate moiety; this embodiment of the method can, for example, facilitate penetration of the antihistamine across the blood-brain barrier such as for autoimmune disease with CNS involvement (e.g., autoimmune demyelinating disease). In one exemplary embodiment, the antihistamine is pyrilamine. The H1-antihistamines not encompassed by the methods provided herein are cyproheptadine and hydroxyzine.

Derivatized Antihistamines: In certain embodiments, the agent can be a derivatized form of a predetermined antihistamine (e.g., derivatives of pyrilamine, brompheniramine, diphenhydramine, fexofenadine, cetrizine, etc.). Derivatives of a predetermined antihistamine are those with a chemical modification of the antihistamine. Such derivatives can be prepared by chemically modifying the predetermined antihistamine using standard chemical methods known in the art. Examples of suitable chemical modifications include addition, removal or substitution of the following substituents:

(1) hydrocarbon substituents, such as aliphatic (e.g. linear or branched alkyl, alkenyl, or alkynyl), alicyclic (e.g., cycloalkyl, or cycloalkenyl) substituents, aromatic, aliphatic and alicyclic-substituted aromatic nuclei, and the like, as well as cyclic substituents;

(2) substituted hydrocarbon substituents, such as those substituents containing nonhydrocarbon radicals which do not alter the predominantly hydrocarbon substituent (e.g., halo (especially bromo, chloro and fluoro), alkoxy, acetyl, carbonyl, mercapto, alkylmercapto, sulfoxy, nitro, nitroso, amino, alkyl amino, amide, and the like);

(3) hetero substituents, that is, substituents which, while having predominantly hydrocarbyl character, contain other than carbon atoms. Suitable heteroatoms include, for example, sulfur, oxygen, hydroxyl, nitrogen, and such substituents as, for example, pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Libraries of antihistamine derivatives can also be prepared by rational design. (See generally, Cho et al., Pac. Symp. Biocompat. 305-16, 1998); Sun et al., J. Comput. Aided Mol. Des. 12:597-604, 1998); each incorporated herein by reference in their entirety). For example, libraries of antihistamine derivatives can be prepared by syntheses of combinatorial chemical libraries (see generally DeWitt et al., Proc. Nat. Acad. Sci. USA 90:6909-13, 1993; International Patent Publication WO 94/08051; Baum, Chem.& Eng. News, 72:20-25, 1994; Burbaum et al., Proc. Nat. Acad. Sci. USA 92:6027-31, 1995; Baldwin et al., J. Am. Chem. Soc. 117:5588-89, 1995; Nestler et al., J. Org. Chem. 59:4723-24, 1994; Borehardt et al., J. Am. Chem. Soc. 116:373-74, 1994; Ohlmeyer et al., Proc. Nat. Acad. Sci. USA 90:10922-26, 1993; and Longman, Windhover's In Vivo The Business & Medicine Report 12:23-31, 1994, all of which are incorporated by reference herein in their entirety.)

The following articles describe methods for selecting starting molecules and/or criteria used in their selection: Martin et al., J. Med. Chem. 38:1431-36, 1995; Domine et al., J. Med. Chem., 37:973-80, 1994; Abraham et al., J. Pharm. Sci. 83:1085-100, 1994; each of which is hereby incorporated by reference in its entirety.

A “combinatorial library” is a collection of compounds in which the compounds comprising the collection are composed of one or more types of subunits. The subunits can be selected from natural or unnatural moieties. The compounds of the combinatorial library differ in one or more ways with respect to the number, order, type or types of modifications made to one or more of the subunits comprising the compounds. Alternatively, a combinatorial library may refer to a collection of “core molecules” which vary as to the number, type or position of R groups they contain and/or the identity of molecules composing the core molecule. The collection of compounds is generated in a systematic way. Any method of systematically generating a collection of compounds differing from each other in one or more of the ways set forth above is a combinatorial library.

A combinatorial library can be synthesized on a solid support from one or more solid phase-bound resin starting materials. The library can contain five (5) or more, preferably ten (10) or more, organic molecules which are different from each other (i.e., five (5) different molecules and not five (5) copies of the same molecule). Each of the different molecules (different basic structure and/or different substituents) is present in an amount such that its presence can be determined by some means (e.g., can be isolated, analyzed, detected with a binding partner or suitable probe). The actual amounts of each different molecule needed so that its presence can be determined can vary due to the actual procedures used and can change as the technologies for isolation, detection and analysis advance. When the molecules are present in substantially equal molar amounts, an amount of 100 picomoles or more can be detected. Preferred libraries comprise substantially equal molar amounts of each desired reaction product and do not include relatively large or small amounts of any given molecules so that the presence of such molecules dominates or is completely suppressed in any assay.

Combinatorial libraries are generally prepared by derivatizing a starting compound onto a solid-phase support (such as a bead). In general, the solid support has a commercially available resin attached, such as a Rink or Merrifield Resin. After attachment of the starting compound, substituents are attached to the starting compound. Substituents are added to the starting compound, and can be varied by providing a mixture of reactants comprising the substituents. Examples of suitable substituents include, but are not limited to, the following:

(1) hydrocarbon substituents, that is, aliphatic (e.g. alkyl or alkenyl), alicyclic (e.g., cycloalkyl, cycloalkenyl) substituents, aromatic, aliphatic and alicyclic-substituted aromatic nuclei, and the like, as well as cyclic substituents;

(2) substituted hydrocarbon substituents, that is, those substituents containing nonhydrocarbon radicals which do not alter the predominantly hydrocarbon substituent (e.g., halo (especially chloro and fluoro), alkoxy, mercapto, alkylmercapto, nitro, nitroso, sulfoxy, and the like);

(3) hetero substituents, that is, substituents which, while having predominantly hydrocarbyl character, contain other than carbon atoms. Suitable heteroatoms include, for example, sulfur, oxygen, nitrogen, and such substituents as pyridyl, furanyl, thiophenyl, imidazolyl, and the like. Heteroatoms, and typically no more than one, can be present for each carbon atom in the hydrocarbon-based substituents. Alternatively, there can be no such radicals or heteroatoms in the hydrocarbon-based substituent and, therefore, the substituent can be purely hydrocarbon.

Derivatized H1-antihistamines typically retain the H1 antagonist pharmacophore having cis- and trans-aromatic rings. Further, where an antihistamine derivative is used according to the methods provided herein, the agent typically retains the carboxylate moiety that can interact with Lys²⁰⁰ of the H1 receptor (see Wieland et al.). In certain embodiments of the invention, for example, those in which the central nervous system is involved in the autoimmune disease (e.g., autoimmune demyelinating disease such as multiple sclerosis or EAE), derivatized antihistamines lacking the carboxylate moiety can be used to facilitate penetration of the agent across the blood-brain barrier.

Methods of making combinatorial libraries are known in the art, and include the following: U.S. Pat. Nos. 5,958,792; 5,807,683; 6,004,617; 6,077,954; which are incorporated by reference herein.

The ability of an antihistamine derivative to bind or block H1R can be assayed using routine methods known in the art. Methods include those directed as assessing the occupancy of the H1 receptor binding site by the derivative compound as well as functional assays. Such methods generally comprise administering the antihistamine derivative to cells that expresses functional H1R. Such cells can, for example, endogenously express H1R (for example, the smooth muscle cell line DDT1MF-2, see, e.g., Mitsuhashi and Payan, J. Cell. Physiol. 134:367-375, 1988). In addition, recombinant H1 receptor displaying the functional and binding characteristics of native H1R can also be used by, for example, stably expressing a cDNA encoding the H1R in a cell line such as, for example, CHO cells (see, e.g., Moguilevsky et al., J. Recept. Signal Transduct. Res. 15:91-102, 1995). Binding of the administered antihistamine derivative to the H1 receptor can be assayed using a labeled derivative compound (e.g., [³H]- or [¹²⁵I]-labeled compound) and measuring the amount of label bound to the cells. Specificity for binding to cellular H1R can be controlled for by, e.g., measuring background binding of the compound to cells not expressing H1R. Also, specificity for the H1R antagonist binding site of cellular H1R can be determined by, for example, the ability of a known H1R antihistamine to compete for H1R binding in a dose-dependent manner. Antibodies specific for the H1-antihistamine binding domain of H1R can also be used to determine specificity of binding by dose-dependent inhibition. (See, e.g., Mitsuhashi and Payan.) The binding characteristics of antihistamine derivatives can be further analyzed at the protein level. For example, cellular protein can be solubilized using, e.g., 1% digitonin, the solubilized proteins purified (e.g., by sequential gel filtration, chromatofocusing, and reverse phase high pressure liquid chromatography), and the derivative-binding protein identified by measuring incorporation of label into the separated polypeptides.

To measure the ability of an antihistamine derivative to block H1R, functional assays such as those known in the art may also be employed. For example, cells expressing functional H1R as described above can be exposed to histamine in the presence or absence of the antihistamine derivative and inhibition of H1R-mediated stimulation of phospholipase C (PLC)-mediated breakdown of polyphosphoinositides determined by measuring [³H]inositol phosphate (IP₃) formation.

Antibodies: In yet another embodiment, the H1R-blocking agent is an H1R-blocking antibody, i.e., an anti-H1R antibody that acts as an H1 receptor antagonist. The H1-antihistamine-binding region of the H1 receptor can be used as an immunogen to generate antibodies which immunospecifically bind to the histamine-binding region of H1R, thereby preventing the interaction of histamine with H1R by direct competition. The histamine binding region and the amino acids in H1R crucial for binding of histamine are known. (See, e.g., Leurs et al., Biochem. Biophys. Res. Commun. 214:110-117, 1995.) The immunogen can, for example, include peptide fragments of H1R comprising these amino acids (e.g., Asp²⁰⁷ and/or Lys²⁰⁰). Such peptides can be generated by, for example, synthetic methods known in the art (see infra). In addition, antibodies can be generated to allosteric sites of H1R to produce antibodies that negatively regulate H1 receptor activity allosterically (e.g., binding to and stabilizing the inactive conformation of the receptor). The binding site for H1-antihistamines, which act as inverse agonists as described supra, is also known, including amino acids crucial for binding to both first and second generation H1-antihistamines (e.g., Asp¹¹⁶, Trp¹⁶⁷, Phe⁴³³, and Phe⁴³⁶) as well as for selective binding to second generation H1-antihistamines containing a carboxylate moiety (Lys²⁰⁰). (See, e.g., Wieland et al.) Immunogens for antibody production can include peptide fragments of the H1 receptor including one or more of these amino acids to generate antibodies that have inhibitory effects on H1 by, for example, stabilizing an inactive conformation of the H1 receptor. Further, peptide fragments for immunization can be, for example, denatured or, alternatively, non-denatured to retain conformational epitopes.

Such antibodies include but are not limited to monoclonal antibodies, chimeric antibodies, single chain antibodies, and heavy chain antibody fragments (e.g., F(ab′), F(ab′)₂, Fv, or hypervariable regions). In preferred embodiments, fragments lacking the F_{c} portion of the antibody (e.g., F_{ab}, Fᵥ) are used to avoid activation of F_{c} receptors on immune cells.

Methods for making and using antibodies are generally disclosed by Harlow and Lane (Using Antibodies, A Laboratory Manual, Cold Spring Harbor Laboratory, Cold Spring Harbor, N.Y. (1999); the disclosure of which is incorporated by reference herein). For preparation of monoclonal antibodies directed toward H1R or a fragment thereof, any technique which provides for the production of antibody molecules by continuous cell lines in culture can also be used. Such techniques include, for example, the hybridoma technique originally developed by Kohler and Milstein (see, e.g., Nature 256:495-97, 1975), as well as the trioma technique, (see, e.g., Hagiwara and Yuasa, Hum. Antibodies Hybridomas 4:15-19, 1993), the human B-cell hybridoma technique (see, e.g., Kozbor et al., Immunology Today 4:72, 1983), and the EBV-hybridoma technique to produce human monoclonal antibodies (see, e.g., Cole et al., In Monoclonal Antibodies and Cancer Therapy, Alan R. Liss, Inc., pp. 77-96, 1985). Human antibodies can be used and can be obtained by using human hybridomas (see, e.g., Cote et al., Proc. Natl. Acad. Sci. USA 80:2026-30, 1983) or by transforming human B cells with EBV virus in vitro (see, e.g., Cole et al., supra). Methods for obtaining human antibodies are also disclosed in Lonberg et al., WO93/12227 (1993), U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994) Nature Biotechnology 14, 826 (1996); and Kucherlapati, WO 91/10741 (1991).

Further, “chimeric” or “humanized” antibodies can be prepared (see, e.g., Morrison et al., Proc. Natl. Acad. Sci. USA 81:6851-5, 1984; Neuberger et al., Nature 312:604-08, 1984; Takeda et al., Nature 314:452-4, 1985). Chimeric antibodies are typically prepared by splicing the non-human genes for an antibody molecule specific for a H1R polypeptide together with genes from a human antibody molecule of appropriate biological activity. It can be desirable to transfer the antigen binding regions (e.g., F(ab′)₂, F(ab′), Fv, or hypervariable regions) of non-human antibodies into the framework of a human antibody by recombinant DNA techniques to produce a substantially human molecule. Methods for producing such “chimeric” molecules are generally well known and described in, for example, U.S. Pat. Nos. 4,816,567; 4,816,397; 5,693,762; and 5,712,120; International Patent Publications WO 87/02671 and WO 90/00616; and European Patent Publication EP 239 400; the disclosures of which are incorporated by reference herein). Alternatively, a human monoclonal antibody or portions thereof can be identified by first screening a human B-cell cDNA library for DNA molecules that encode antibodies that specifically bind to an H1R polypeptide according to the method generally set forth by Huse et al. (Science 246:1275-81, 1989). The DNA molecule can then be cloned and amplified to obtain sequences that encode the antibody (or binding domain) of the desired specificity. Phage display technology offers another technique for selecting antibodies that bind to H1R polypeptides or fragments thereof. (See, e.g., International Patent Publications WO 91/17271 and WO 92/01047; and Huse et al., supra). Methods for making humanized antibodies are also disclosed in Queen et al., Proc. Natl. Acad. Sci. USA 86:10029-10033, 1989; and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539.

Techniques described for the production of single chain antibodies (see, e.g., U.S. Pat. Nos. 4,946,778 and 5,969,108) can be adapted to produce H1R-specific single chain antibodies. An additional aspect of the invention utilizes the techniques described for the construction of a F_{ab} expression library (see, e.g., Huse et al. (1989) supra) to allow rapid and easy identification of monoclonal F_{ab} fragments with the desired specificity for H1R polypeptides or fragments thereof.

The immunoglobulins also can be heavy chain antibodies. Immunoglobulins from animals such as camels, dromedaries, and llamas (Tylopoda) can form heavy chain antibodies, which comprise heavy chains without light chains. (See, e.g., Desmyter et al., J. Biol. Chem. 276:26285-90, 2001; Muyldermans and Lauwereys, J. Mol. Recognit. 12:131-40, 1999; Arbabi Ghahroudi et al., FEBS Lett. 414:521-26, 1997; Muyldermans et al., Protein Eng. 7:1129-35, 1994; Hamers-Casterman et al., Nature 363:446-48, 1993; the disclosures of which are incorporated by reference herein.) The variable region of heavy chain antibodies are typically referred to as “VHH” regions. (See, e.g., Muyldermans et al., TIBS 26:230-35, 2001.) The VHH of heavy chain antibodies typically have enlarged or altered CDR regions, as such enlarged CDR1 and/or CDR3 regions. Methods of producing heavy chain antibodies are also known in the art. (See, e.g., Arbabi Ghahroudi et al.; Muyldermans and Lauwereys.)

In addition F_{ab} or Fᵥ fragments of H1R antibodies can be produced using, for example, recombinant techniques known in the art. (See, e.g., the methods described in U.S. Pat. No. 5,965,405 for the recombinant production of Fᵥ fragments.)

In the production of antibodies, screening for the desired antibody can be accomplished by techniques known in the art (e.g., ELISA). For example, a specific H1R peptide fragment containing the histamine binding region can be used to assay generated hybridomas for a product which binds to that peptide. In addition, the antibodies can be evaluated in functional assays of H1R activation known in the art such as, for example, assays for breakdown of polyphosphoinositides as measured by, e.g., [³H]inositol phosphate (IP₃) formation, as noted supra.

Peptides: In one embodiment, the H1R-blocking agent is a peptide. The peptide can act as a competitive inhibitor of histamine for binding to H1R by specifically binding the histamine-binding site on H1R (e.g., binding to Asp²⁰⁷ and or Lys²⁰⁰ of H1R). Alternatively, the peptide can, for example, act allosterically by stabilizing an inactive conformation of H1R; such peptides can, for example, be designed to interact with the binding site in H1R for H1-antihistamines (e.g., the peptide can interact with Asp¹¹⁶, Trp¹⁶⁷, Phe⁴³³, Phe⁴³⁶, and/or Lys²⁰⁰). Generally, peptide agents encompassed by the methods provided herein range in size from about 3 amino acids to about 100 amino acids, with peptides ranging from about 3 to about 25 being typical and with from about 3 to about 12 being more typical. Peptide agents can be synthesized by standard chemical methods known in the art (see, e.g., Hunkapiller et al., Nature 310:105-11, 1984; Stewart and Young, Solid Phase Peptide Synthesis, 2^{nd} Ed., Pierce Chemical Co., Rockford, Ill., (1984)), such as, for example, an automated peptide synthesizer. In addition, such peptides can be produced by translation from a vector having a nucleic acid sequence encoding the peptide using methods known in the art (see, e.g., Sambrook et al., Molecular Cloning, A Laboratory Manual, 3rd ed., Cold Spring Harbor Publish., Cold Spring Harbor, N.Y. (2001); Ausubel et al., Current Protocols in Molecular Biology, 4th ed., John Wiley and Sons, New York (1999); which are incorporated by reference herein).

Peptide libraries can be constructed from which H1R-blocking peptides can be determined. The library can comprise synthetic peptides. For example, a population of synthetic peptides representing all possible amino acid sequences of length N (where N is a positive integer), or a subset of all possible sequences, can comprise the peptide library. Such peptides can be synthesized by standard chemical methods known in the art (see, e.g., Hunkapiller et al., Nature 310:105-11, 1984; Stewart and Young, Solid Phase Peptide Synthesis, 2^{nd} Ed., Pierce Chemical Co., Rockford, Ill., (1984)), such as, for example, an automated peptide synthesizer. Furthermore, if desired, nonclassical amino acids or chemical amino acid analogs can be used in substitution of or in addition into the classical amino acids. Non-classical amino acids include but are not limited to the D-isomers of the common amino acids, α-amino isobutyric acid, 4-aminobutyric acid, 2-amino butyric acid, γ-amino butyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, selenocysteine, fluoro-amino acids, designer amino acids such as β-methyl amino acids, C α-methyl amino acids, N α-methyl amino acids, and amino acid analogs in general. Furthermore, the amino acid can be D (dextrorotary) or L (levorotary).

Peptide libraries can also be produced by transcription and translation from a library of nucleic acid sequences. For example, oligonucleotide libraries can be produced from fragments of genomic DNA and/or cDNA from a particular organism. Methods of making randomly sheared genomic DNA and/or cDNA, and of manipulating such DNAs, are known in the art. (See Sambrook et al., supra; Ausubel et al., supra.) Also, a random peptide library can be produced from a population of synthetic oligonucleotides encoding all possible amino acid sequences of length N (where N is a positive integer), or a subset of all possible sequences. Alternatively, a semi-random library can be used. For example, a semi-random library can be designed according to the codon usage preference of the host cell or to minimize the inclusion of translational stop codons in the encoded amino acid sequence. As an example of the latter, in the first position of each codon, equimolar amounts of C, A, and G and a one half-molar amount of T would be used. In the second position, A is used at a one half-molar amount while C, T, and G would be used in equimolar amounts. In the third position, only equimolar amounts of G and C would be used. Methods of making synthetic DNA are known to those of skill in the art. (See, e.g., Glick and Pasternak, Molecular Biotechnology: Principles and Applications of Recombinant DNA, ASM Press, Washington, D.C., 1998.) Such a collection of oligonucleotides can be directly ligated into a vector, into an expression vector (i.e., a vector that includes specific cis regulatory sequences in an expression cassette to effect expression of nucleic acid inserts; see infra), and the like. Procedures for creating peptide expression libraries are well known in the art. (See, e.g., Ausubel et al., supra; Sambrook et al., supra.)

To determine H1R-blocking peptides for use in the methods described herein, candidate peptides can be evaluated for their ability to bind the H1 receptor and downmodulate receptor activation. The pharmacophore for H1-antihistamines (see Wieland et al.) can be utilized in structure-based design methods known in the art (see, e.g., Kuntz et al., J. Mol. Biol. 161:269-288, 1982) to “fit” small putative peptides, including peptides having non-classical amino acids, three dimensionally into appropriate sites on the H1 receptor. Best-scoring peptides can then be produced, for example, synthetically as described above to generate a small peptide library for evaluation in standard binding and functional assays known in the art (see, e.g., methods described supra).

In addition, methods are known in the art for constructing peptide expression libraries wherein the peptides are presented extracellularly on the cell surface of the host cells. (See U.S. Pat. No. 6,153,380, incorporated by reference herein.) Using these methods, peptides, expressed in host cells having, e.g. a reporter gene for H1 receptor activation, can be evaluated functionally for their ability to block histamine-mediated activation of the H1 receptor stably expressed on the host cells. Cells expressing H1R-blocking peptides can be expanded and the vector insert encoding the peptide subcloned and sequence using known methods (see, e.g., Ausubel et al., supra; Sambrook et al., supra.)

### Administration of H1R-Blocking Agents to Subjects

As noted above, the methods of the invention involve administering an effective amount of a H1R-blocking agent to a subject suffering from, or at elevated risk of developing, an autoimmune disease. In each of the embodiments of the invention described herein, the H1R-blocking agent (e.g., antihistamine, antibody, peptide) is delivered in a manner consistent with conventional methodologies associated with the management of the autoimmune disorder for which treatment or prevention is sought. In accordance with the disclosure herein, an effective amount of the agent is administered to a subject in need of such treatment for a time and under conditions sufficient to prevent or treat the autoimmune reactions.

Subjects for H1R-blocking therapy according to the invention include patients at high risk for developing an autoimmune disease as well as patients presenting with existing autoimmune disease. Typically, the subject has been diagnosed as having an autoimmune disease for which treatment or prevention is sought. Further, subjects can be monitored during the course of the treatment for any change in autoimmune disease symptoms in response to the treatment. Also, in certain embodiments of the invention, the subject does not suffer from another disease or disorder that requires treatment involving H1 receptor-blockade.

To identify subject patients for prevention or treatment according to the methods of the invention, accepted screening methods are employed to determine risk factors associated with specific autoimmune disorders or to determine the status of an existing disorder identified in a subject. Such methods can include, for example, determining whether an individual has relatives who have been diagnosed with an autoimmune disease. Screening methods can also include, for example, conventional work-ups to determine familial status for a particular autoimmune or inflammatory disease known to have a heritable component. Toward this end, nucleotide probes can be routinely employed to identify individuals carrying genetic markers associated with a particular autoimmune disease of interest. In addition, a wide variety of immunological methods are known in the art that are useful to identify markers for specific autoimmune diseases. For example, various ELISA immunoassay methods are available and well-known in the art that employ monoclonal antibody probes to detect autoantibodies associated with specific physiological markers of autoimmune disease. Such screening may be implemented as indicated by known patient symptomology, age factors, related risk factors, etc. These methods allow the clinician to routinely select patients in need of the methods described herein for prevention or treatment of autoimmune disease. In accordance with these methods, H1R-blocking therapy may be implemented as an independent prevention or treatment program or as a follow-up, adjunct, or coordinate treatment regimen to other treatments.

The H1R-blocking agent is formulated with a pharmaceutically acceptable carrier and administered in an effective amount, i.e., sufficient to modulate the autoimmune response and inhibit initiation or progression of the autoimmune disease in the subject. According to the method of the present invention, the agent may be administered to subjects by a variety of administration modes, including, for example, by intramuscular, subcutaneous, intravenous, intra-atrial, intra-articular, parenteral, intranasal, intrapulmonary, transdermal, and oral routes of administration. For prevention and treatment purposes, the agent may be administered to a subject in a single bolus delivery, via continuous delivery (e.g., continuous transdermal delivery) over an extended time period, or in a repeated administration protocol (e.g., on an hourly, daily, or weekly basis). One preferred embodiment is the oral route of administration. Antihistamine drugs are traditionally administered orally for other disease conditions (e.g., allergy). Acceptable formulations for administration of antihistamines by the oral route are well-known in the art and can be adapted for the methods provided herein.

The various dosages and delivery protocols contemplated for administration of the H1R-blocking agents are effective to inhibit the occurrence or ameliorate one or more symptoms of the target autoimmune response in the subject. Determination of effective dosages in this context is typically based on animal model studies followed up by human clinical trials and is guided by determining effective dosages and administration protocols that significantly reduce the occurrence or severity of the subject autoimmune disease in model subjects.

The actual dosage of the H1R-blocking agent can vary according to factors such as the disease state, age, and weight of the individual subject, as well as the specific activity of the agent itself and its ability to elicit the desired response in the individual. Dosage regimens may be adjusted to provide an optimum therapeutic response. A therapeutically effective amount is also one in which any undesired collateral effects are outweighed by beneficial effects of inhibiting the autoimmune response. For embodiments in which the agent is an FDA-approved H1-antihistamine drug, because such drugs are well-known in the medical practice for other disease conditions (e.g., allergy, asthma), many of the collateral effects at various dosage ranges have already been determined. A non-limiting range for a an effective amount of the agent is about 1 μg/kg to about 35 mg/kg, and in more specific embodiments between about 1 μg/kg and about 20 mg/kg, between about 10 μg/kg and about 10 mg/kg, or between about 0.1 mg/kg and about 5 mg/kg Dosages within this range can be achieved by single or multiple administrations, including, e.g., multiple administrations per day or daily or weekly administrations. Also, for embodiments in which the agent is an antihistamine, the upper value for the dosage range can further depend on the LD₅₀ value for the antihistamine. LD₅₀ values for known drugs, including antihistamines, are known and are available, for example, in the The Merck Index—An Encyclopedia of Chemicals, Drugs, and Biologicals (Maryadele J. O'Neil et al. eds., Merck & Co., 13th ed. 2001), incorporated by reference herein in its entirety.

Dosage of the H1R-blocking agent may be varied by the attending clinician to maintain a desired concentration at the target site. For example, it an intravenous mode of delivery is selected, local concentration of the agent in the bloodstream at the target tissue may be between about 1-50 nanomoles of the agent per liter, sometimes between about 1.0 nanomole per liter and 10, 15, or 25 nanomoles per liter depending on the subject's status and projected measured response. Higher or lower concentrations may be selected based on the mode of delivery, e.g., trans-epidermal delivery versus delivery to a mucosal surface. Dosage should also be adjusted based on the release rate of the administered formulation, e.g., nasal spray versus powder, sustained release oral or injected particles, transdermal formulations, etc. To achieve the same serum concentration level, for example, slow-release particles with a release rate of 5 nanomolar (under standard conditions) would be administered at about twice the dosage of particles with a release rate of 10 nanomolar.

Co-Administration of H1R-Blocking Agents with a Second Active Agent

In certain embodiments of the invention, the methods for treating or preventing an autoimmune disease include co-administration of the H1R-blocking agent with a second active agent. Second active agents used for co-administration typically include pharmacological agents that can downmodulate the immune response against self either alone or in conjunction with H1R blockade. Such agents include those that are typically used for the treatment of an autoimmune disease. In certain embodiments, the second active agent is not a dithiocarbamate disulfide derivative; a substituted 1,4-dihydropyridine bradykinin antagonist; a heteroaryl substituted 1,4-dihydropyridine bradykinin antagonist; a LTB-receptor antagonist comprising disubstituted phenyl-benzamidine derivative; or a small molecule antagonist of chemokine receptor CCR1, each as defined supra. Examples of second active agents that can be used for co-administration according to the methods provided herein are described below.

Self-vectors: In one embodiment of the invention, the H1 receptor-blocking agent (e.g., antihistamine) is co-administered with a self-vector encoding a self-polypeptide associated with the disease for which treatment or prevention is desired. Self-vectors for use according to the methods provided herein, including examples of encoded self-polypeptides and methods for administration in the treatment or prevention of autoimmune disease, are described in U.S. patent application Ser. No. 10/302,098, incorporated by reference herein.

In one embodiment of the invention, the autoimmune disease is multiple sclerosis and the self-vector co-administered with the H1R-blocking agent encodes one or more of the following self-polypeptides: myelin basic protein (MBP), proteolipid protein (PLP), myelin associated glycoprotein (MAG), cyclic nucleotide phosphodiesterase (CNPase), myelin-associated oligodendrocytic basic protein (MBOP), myelin oligodendrocyte protein (MOG), or alpha-B crystalline.

In another embodiment, the autoimmune disease is insulin dependent diabetes mellitus and the self-vector co-administered with the H1R-blocking agent encodes one or more of the following self-polypeptides: insulin, insulin B chain, preproinsulin, proinsulin, glutamic acid decarboxylase 65 kDa and 67 kDa forms, tyrosine phosphatase IA2 or IA-2b, carboxypeptidase H, heat shock proteins, glima 38, islet cell antigen 69 kDa, p52, or islet cell glucose transporter (GLUT 2).

In other embodiments of the invention, co-administration of the self-vector and H1R-blocking agent can include administration with a polynucleotide having an immune modulatory sequence (IMS). As note supra, IMSs may be oligonucleotides or a sequence of nucleotides incorporated in a vector. IMSs, examples thereof, and their use in conjunction with self-vectors for treating or preventing autoimmune disease are also described in U.S. patent application Ser. No. 10/302,098. In certain embodiments, the IMS is 5′-Purine-Pyrimidine-[X]-[Y]-Pyrimidine-Pyrimidine-3′ or 5′-Purine-Purine [X]-[Y]-Pyrimidine-Pyrimidine-3′, wherein X and Y are any naturally occurring or synthetic nucleotide, except that X and Y cannot be cytosine-guanine.

In yet another embodiment of the present invention, co-administration of the self-vector and H1R-blocking agent can include administration with an immunomodulatory protein or vector encoding an immunomodulatory protein as described below. Co-administration of self-vectors with immunomodulatory proteins, including cytokines and chemokines, or vectors encoding them are also described in U.S. patent application Ser. No. 10/302,098.

Immunomodulatory Proteins and vectors encoding immunomodulatory proteins: In one embodiment of the invention, administration of the H1 receptor-blocking agent (e.g., antihistamine) includes co-administered with an immunomodulatory protein (e.g., a cytokine such as IL-4, IL10, IL-13, or the like; Ig fusions of costimulatory molecules such as CTLA-4; etc.); in other embodiments, administration of the H1 receptor-blocking agent (e.g., antihistamine) includes co-administered with a vector encoding the immunomodulatory protein. In certain embodiments, the immunomodulatory protein can exert an effect on balance of Th1/Th2 pathways of the immune response. Thus, for example, where Th1 pathways are implicated in an autoimmune disease, a Th2 cytokine (such as, for example, IL-4) can be co-administered or vice versa.

To avoid the possibility of eliciting unwanted anti-self immunomodulatory protein (e.g., anti-self cytokine) responses when using immunomodulatory protein co-delivery, chemical (small molecule) immunodulatory agents such as the active form of vitamin D3 can also be used. In this regard, 1,25-dihydroxy vitamin D3 has been shown to exert an adjuvant effect via intramuscular DNA immunization.

As noted above, polynucleotide sequences coding for immunomodulatory proteins (e.g., cytokines, costimulatory molecules) can be coadministered with the H1 receptor-blocking agent. Thus, genes encoding one or more immunomodulatory protein or functional fragments thereof (for example, one of the various cytokines such as the interleukins, interferons, or colony stimulating factors) may be used in the instant invention. The gene sequences for a number of these proteins are known.

In one embodiment of the invention, the immunomodulatory protein co-administered with the H1R-blocking agent is a cytokine or chemokine. In other embodiments, a vector encoding the cytokine or chemokine is co-administered. In certain embodiments, the cytokine is IL-4, IL-10, or IL-13.

Nucleotide sequences selected for use in the present invention can be derived from known sources, for example, by isolating the nucleic acid from cells containing a desired gene or nucleotide sequence using standard techniques. Similarly, the nucleotide sequences can be generated synthetically using standard modes of polynucleotide synthesis that are well known in the art. See, e.g., (Edge et al., Nature 292:756, 1981; Nambair et al., Science 223:1299, 1984; Jay et al., J. Biol. Chem. 259:6311, 1984. Generally, synthetic oligonucleotides can be prepared by either the phosphotriester method as described by (Edge et al.; Duckworth et al., Nucleic Acids Res. 9:1691, 1981, or the phosphoramidite method as described by (Beaucage et al., Tet. Letts. 22:1859, 1981), and Matteucci et al., J. Am. Chem. Soc. 103:3185, 1981). Synthetic oligonucleotides can also be prepared using commercially available automated oligonucleotide synthesizers known in the art (see supra). The nucleotide sequences can thus be designed with appropriate codons for a particular amino acid sequence. In general, one selects preferred codons for expression in the intended host. The complete sequence is assembled from overlapping oligonucleotides prepared by standard methods and assembled into a complete coding sequence. See, e.g., Edge et al.; Nambair et al., Jay et al.

Another method for obtaining nucleic acid sequences for use herein is by recombinant means. Thus, a desired nucleotide sequence can be excised from a plasmid carrying the nucleic acid using standard restriction enzymes and procedures. Site specific DNA cleavage is performed by treating with the suitable restriction enzymes and procedures. Site specific DNA cleavage is performed by treating with the suitable restriction enzyme (or enzymes) under conditions which are generally understood in the art, and the particulars of which are specified by manufacturers of commercially available restriction enzymes. If desired, size separation of the cleaved fragments may be performed by polyacrylamide gel or agarose gel electrophoreses using standard techniques.

Yet another convenient method for isolating specific nucleic acid molecules is by the polymerase chain reaction (PCR). (Mullis et al., Methods Enzymol. 155:335-350 1987).

Vector systems and methods for delivering nucleic acid preparations are known in the art. See, e.g., U.S. Pat. Nos. 5,399,346, 5,580,859, 5,589,466. A number of viral based systems have been developed for transfer into mammalian cells. For example, retroviral systems have been described (U.S. Pat. No. 5,219,740; Miller et al., Biotechniques 7:980-990, 1989; Miller, Human Gene Therapy 1:5-14, 1990; Scarpa et al., Virology 180:849-852, 1991; Burns et al., Proc. Natl. Acad. Sci. USA 90:8033-8037, 1993; Boris-Lawrie and Temin, Cur. Opin. Genet. Develop. 3:102-109, 1993). A number of adenovirus vectors have also been described, see e.g., Haj-Ahmad et al., J. Virol. 57:267-274, 1986; Bett et al., J. Virol. 67:5911-5921, 1993; Mittereder et al., Human Gene Therapy 5:717-729, 1994; Seth et al., J. Virol. 68:933-940, 1994; Barr et al., Gene Therapy 1:51-58, 1994; Berkner, BioTechniques 6:616-629, 1988; Rich et al., Human Gene Therapy 4:461-476, 1993. Adeno-associated virus (AAV) vector systems have also been developed for nucleic acid delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al., Molec. Cell. Biol. 8:3988-3996, 1988; Vincent et al., Vaccines 90 (Cold Spring Harbor Laboratory Press) 1990; Carter, Current Opinion in Biotechnology 3:533-539, 1992; Muzyczka, Current Topics in Microbiol. And Immunol. 158:97-129, 1992; Kotin, Human Gene Therapy 5:793-801, 1994; Shelling et al., Gene Therapy 1:165-169, 1994; and, Zhou et al., J. Exp. Med. 179:1867-1875, 1994).

The polynucleotide can also be delivered without a viral vector. For example, the molecule can be packaged in liposomes prior to delivery to the subject. Lipid encapsulation is generally accomplished using liposomes which are able to stably bind or entrap and retain nucleic acid. For a review of the use of liposomes as carriers for delivery of nucleic acids, see Hug et al., Biochim. Biophys. Acta. 1097:1-17, 1991; Straubinger et al., in Methods of Enzymology, Vol. 101, pp. 512-527, 1983.

Methods for administration of polynucleotides encoding immunomodulatory proteins are further described in U.S. patent application Ser. No. 10/302,098.

A further understanding of the present invention will be obtained by reference to the following description that sets forth illustrative embodiments.

### Example 1

### Induction of Experimental Autoimmune Encephalomyelitis (EAE)

EAE was induced with PLP 139-151 in 8 to 12 week old SJL mice (The Jackson Laboratory) as described in Pedotti et al., Nat. Immunol. 2:216-222, 2001. Mice were assessed daily for clinical signs of EAE (see id.). For each mouse, a remission was defined as decrease of the score of at least one point for at least 2 consecutive days. For RNA extraction and transcription analysis, animals were euthanized at different time points during the course of EAE and brains and spinal cords were removed and kept frozen at −80° C. until use. In the pharmacological studies, the histamine receptor 1 antagonist, pyrilamine (Sigma, St Louis, Mo.) and the PAF antagonist, CV6209 (Biomol, Plymouth Meeting, Pa.) were injected daily i.p. in PBS starting 2 days after the induction of EAE. In FcγRIII−/− and +/+, and in FcR γ chain−/− and +/+mice, EAE was induced with MOG35-55 as described in Lock et al, Nat. Med. 8:500-508, 2002. For each mouse with EAE a complete remission was defined as absence of disease for at least 2 consecutive days. Blood was collected from the tail 6 weeks after the immunization and analyzed for antibody responses. Mice were challenged with i.p. injection of 0.1 mg of MOG 35-55 six weeks after the induction of EAE and the presence of anaphylactic reactions was evaluated by measurement of body temperature with a rectal probe (Physitemp, Clifton, N.J.) (see Pedotti et al.).

All animal protocols were approved by the IACUC and the Division of Laboratory Medicine at Stanford, in conformance with NIH guidelines.

### Example 2

### Expression of Allergy-Related Genes in the Central Nervous System in an Autoimmune Demyelinating Disease Model—Concordance with Multiple Sclerosis

In order to assess whether we could utilize the animal model of MS, EAE, to understand the pathobiology of the proteins encoded by “allergy” related genes whose transcripts were elevated in the human MS samples examined in our previous studies (see Table 1), we first analyzed the transcription profiles of these genes in brain and spinal cord of mice with EAE. A relapsing-remitting model of EAE was induced in SJL mice (H-2s) with myelin proteolipid protein (PLP) peptide 139-151 in complete Freund's adjuvant (CFA), and animals were scored daily for clinical signs of disease (see Pedotti et al.). Brain and spinal cord were removed during the acute phase, remissions or relapses of EAE, and RNA was extracted and analyzed by real time quantitative PCR (see Gentle et al., Biotechniques 31:502, 504-506, 508, 2001; Rajeevan et al., J. Mol. Diagn. 3:26-31, 2001).

TABLE 1
Genes related to allergy up regulated in MS

| | | Human genomic | Mouse genomic | |
|---|---|---|---|---|
| Accession number | Entrez Definition | location | location | Reference |
| D10202 | Platelet-activating | 1p35-p34.3 | 4 D2.2 | Lock et al, 2002 |
| | factor receptor | | | |
| M33493 | Tryptase-III | 16p13.3 | 17 | Lock et al, 2002 |
| M89796 | High affinity IgE | 11q12.3 | 19 | Lock et al, 2002 |
| | receptor β chain | | | |
| | gene | | | |
| Z34897 | H1 histamine | 3p25 | 6 | Lock et al, 2002 |
| | receptor | | | |
| M61901 | Prostaglandin D | 9q34.2-34.3 | 2 | Chabas et al, 2001 |
| | synthase | | | |

Quantitative PCR to Assess Target Gene Expression: Brain and spinal cord from were homogenized in Trizol solution (Invitrogen, Carlsbad, Calif.) and RNA was isolated according to the manufacturers instructions under RNase free conditions. RNA was resuspended in 200-500 μl of DEPC treated water and stored at −80° C. until use. RNA was reverse transcribed to cDNA using Superscript II reverse transcriptase (Invitrogen). Briefly, 3 μg of RNA was mixed with reaction mix (final concentration: 1×RT buffer (Invitrogen), 0.5 mM each dNTP (Invitrogen), 100 ng random hexamer (Invitrogen) and DEPC treated water to 20 μl). After a 5 minute incubation at 65° C. followed by chilling on ice, 200 units of Superscript II was added and the mixture was incubated at 25° C. for 10 minutes, 42° C. for 50 minutes, and 70° C. for 15 minutes. cDNA was stored at −20° C. until use.

Expression levels of target genes were analyzed by quantitative PCR using a Lightcycler (Roche, Indianapolis, Ind.). Primer sequences are shown below. Primers for multi-exongenes were designed to span introns and be RNA specific (MMCP-7, PGDS and Actin). To ensure RNA specificity, the primers were optimized on template from RT reactions with or without reverse transcriptase enzyme (data not shown). The primers were used as follows: 1 ul cDNA from the Superscript II reaction was mixed with a final concentration of 1× Quantitect SYBR green reagent (Qiagen, Valencia, Calif.), 1 μM forward primer, 1 μM reverse primer, and DEPC-treated water in a total volume of 20 μl. The PCR conditions for H1R, H2R, PGDS, MMCP-7 and PAFR were as follows: activation at 95° C. for 15 seconds followed by 60 cycles of 94° C. for 15 seconds, 54° C. for 20 seconds, and 72° C. for 19 seconds. A melting curve of the PCR product was obtained by heating at 65° C. for 15 seconds, then increasing to 95° C. at a rate of 0.1° C./second while recording SYBR green fluorescence. The PCR conditions for Actin differed in that the annealing temperature was 55° C. and the extension time was 12 s. Quantification was performed using the relative standard curve method (PE Applied Biosystems, User Bulletin #2, 1997).

| Gene | Accession # | Primer | Sequence |
|---|---|---|---|
| Beta actin | X03 672 | F | GAACCCTAAGGCCAACGCT |
| | | | (SEQ ID NO: 1) |
| | | R | CACGCACGATTTCCCTCTC |
| | | | (SEQ ID NO: 2) |
| H1R | AF387892 | F | TTGAACCGAGAGCGGA |
| | | | (SEQ ID NO: 3) |
| | | R | TGCCCTTAGGAACGAAT |
| | | | (SEQ ID NO: 4) |
| H2R | NM_008286 | F | TGGCACGGTTCATTCC |
| | | | (SEQ ID NO: 5) |
| | | R | GCAGTAGCGGTCCAAG |
| | | | (SEQ ID NO: 6) |
| PAFR | AF004858 | F | CTACAACGAGGGCGAC |
| | | | (SEQ ID NO: 7) |
| | | R | GGGACAAAGAGATGCCA |
| | | | (SEQ ID NO: 8) |
| PDGS | D88329 | F | CTGGTTCCGGGAGAAG |
| | | | (SEQ ID NO: 9) |
| | | R | AGCGTACTCGTCATAGTT |
| | | | (SEQ ID NO: 10) |
| MMCP-7 | L00653 | F | ACACGAGAAGGCATTG |
| | | | (SEQ ID NO: 11) |
| | | R | AGGTACTGCTTACGGAG |
| | | | (SEQ ID NO: 12) |

Transcriptional Profiles of Several Allergy-Related Genes in the Central Nervous System of Mice with EAE are Concordant with Findings in MS Lesions: Mouse mast cell protease-7 (MMCP-7), platelet activating factor receptor (PAFR), and lipocalin-type prostaglandin D synthase (PGDS) were all detected and quantified in brain and spinal cord tissues of EAE-induced mice as described below.

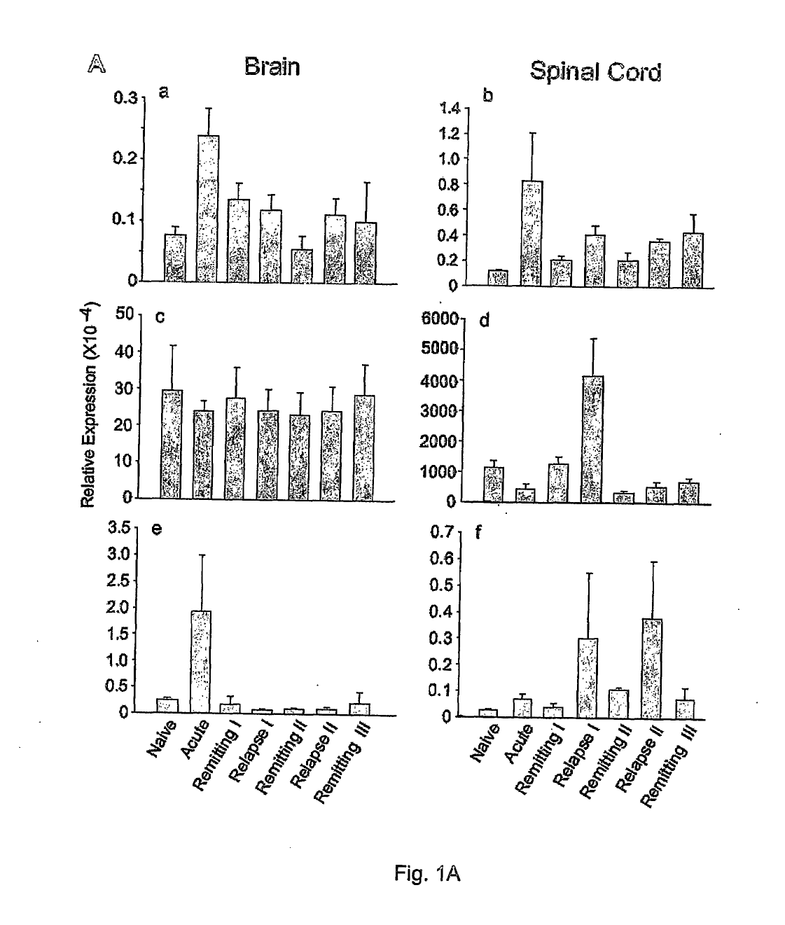

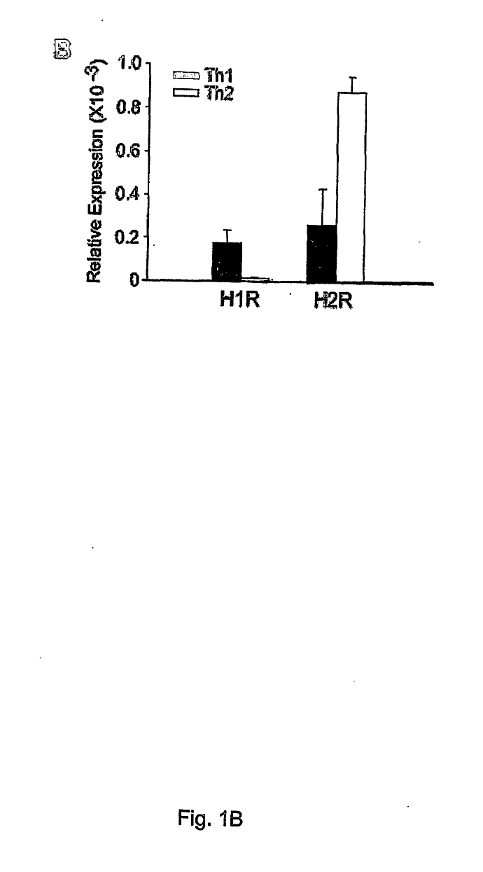

PAF plays a major role in murine anaphylaxis, where, depending on the conditions of immunization and antigen challenge, the role of the IgG1-Fcγ RIII-macrophage-PAF axis can be more important than that of the IgE-FcεRI-mast cell-histamine axis (see Miyajima et al., J. Clin. Invest. 99:901-914, 1997; Strait et al., J. Allergy Clin. Immunol. 109658-668, 2002; Choi et al., J. Exp. Med. 188:1587-1592, 1998). [“Axis” here implies a “pathway” involving the named participants]. PAF may also contribute to anaphylaxis in man (see Strait et al.). Moreover, PAF may have a role in MS. In the cerebrospinal fluid and plasma of patients with the relapsing-remitting form of MS, PAF is elevated and its level correlates with the number of gadolinium MRI enhancing lesions in the brain (Callea et al., Ann. Neurol. 37:63-66, 1995). Quantitative PCR studies showed that PAFR transcripts were elevated 3 and 6 fold in brain and spinal cord, respectively, in the acute phase of EAE compared to naïve mice (p=0.00006 in brain and p=0.03 in spinal cord by ANOVA for acute vs naïve), and remained elevated throughout the course of the disease (FIG. 1a,b). Interestingly, transcripts for PAFR decreased in spinal cord during the remission phase of the disease and increased during the relapsing phase (3 fold in the first relapse, p=0.005 by ANOVA; 3 fold in the second relapse, p=0.0001 by ANOVA) suggesting a role for PAFR in the pathogenesis of a relapse.

Prostaglandin D2 (PGD2) is a major lipid mediator released from mast cells in the late phase of allergic reactions (Fujitani et al., J. Immunol 168:443-449, 2002) and is involved in the regulation of allergic inflammation (see Matsuoka et al., Science 287:2013-2017, 2000). In the brain, PGD2 is also involved in sleep-induction (see Hayaishi, FASEB J. 5:2575-2581, 1991). In a murine asthma model, mice transgenic for lipocalin-type PGD synthase (PGDS) overproduce PGD2, resulting in increased levels of Th2 cytokines and enhanced accumulation of eosinophils and lymphocytes in the lung (Fujitani et al.). PGD2 is also preferentially produced by hematopoietic-PGDS in antigen stimulated human Th2 cells but not Th1 cells (Tanaka et al., J. Immunol. 164:2277-2280, 2000). Although the expression pattern of L-PGDS does not change in brain tissue from EAE animals, where there is already a high background level due to its pleiotropic functions in brain (FIG. 1c), a significant upregulation occurs in the spinal cord during the relapse phase (FIG. 1d) (3.6 fold increase in the first relapse compared to naïve, p=0.013 by ANOVA). Accordingly, PODS may have a role in initiating the relapsing phase of disease.

Mouse mast cell protease-7 is a mouse homologue of human tryptase III (McNeil et al., Proc. Natl. Acad. Sci. USA 89:11174-11178, 1992), which was found to be upregulated in acute MS plaques (Lock et al.). Tryptase has also been shown to be elevated in CSF of patients with MS (13). MMCP-7 is predominantly expressed by mast cells (McNeil et al.; Stevens et al., Proc. Natl. Acad. Sci. USA 91:128-132). In V3 mice with mastocytosis, after sensitization with IgE and subsequent challenge with antigen, MMCP-7 may contribute to anaphylaxis (see Ghildyal et al., J. Exp. Med. 184:1061-1073, 1996). MMCP-7 is significantly upregulated in brain (8 fold) and spinal cord (3 fold) in the acute phase of EAE (p=0.009 and p=0.008 by ANOVA for acute vs naïve in brain and spinal cord FIG. 1e,f, respectively). Relapsing animals also showed increased expression of MMCP-7 in the spinal cord (13 fold during the first relapse and 16 fold during the second one; p=0.08 and p=0.045 for the first and second relapse, respectively, vs. naïve). At least one in vivo substrate of MMCP-7 is believed to be fibrinogen (see Huang et al., J. Biol. Chem. 272:31885-31893, 1997). Perivascular fibrinogen/fibrin deposits are found in EAE and inflammatory MS lesions (Sobel et al., Am. J. Pathol. 131:547-558, 1988; Sobel and Mitchell, Am. J. Pathol 135:161-168, 1989). Interestingly, dermatan sulfate and batroxobin, which degrade fibrinogen and suppress fibrin deposition, respectively, were shown to ameliorate EAE (see Inaba et al., Cell. Immunol 198:96-102, 1999; Inoue et al., J. Neuroimmunol. 71:131-137, 1996).

### Example 3

### Demonstration of IgE Pathways in the Autoimmune Response of EAE

FcγRIII and FcR γ chain-knockout mice: The production of mice with targeted mutations that result in failure of production of the cc chain of the FcγRIII (FcγIII−/− mice) (18) or the FcR γ chain (FcR γ chain−/− mice) (Takai et al., Cell 76:519-529, 1994), and many of the phenotypic characteristics of these mice, have been described in detail. For these studies, we used 8 to 12 week old female Fcγ RIII−/− mice that were backcrossed for six generations with C57B1/6 mice, and used C57B1/6 mice as Fcγ RIII +/+ mice. Female FcR γ chain−/− and +/+ mice were generated by breeding the F₂ offspring of crosses between chimeras and C57BL/6 mice (see, e.g., Lock et al., Nat. Med. 8:500-508; Takai et al.; Miyajima et al., J. Clin. Invest. 99:901-914, 1997). All these mice were purchased from The Jackson Laboratory, Bar Harbor, Me.

Preparation of Tissue Samples for Histological Evaluation: for histological evaluation of EAE in the different knockout mice, 3 to 7 animals per group were sacrificed 6 weeks after the induction of EAE and brain and spinal cord were removed and fixed in 10% formalin. 4-6 micron sections were prepared from paraffin embedded tissues and analyzed (as described in Chabase et al., Science 294:1731-1735, 2001) for inflammatory lesions after hematoxylin and eosin staining by an observer unaware of the identity of individual sections (R.S.).

Measurement of Serum Ig Responses: Peptide-specific IgG1 and IgG2a antibodies were measured in mouse serum samples by ELISA as described in Slavin et al., Autoimmunity 28:109-120, 1998. Briefly, for IgG1 and IgG2a ELISA, 96-wells microtiter plates (Nunc MaxiSorp, Roskilde, Denmark) were coated overnight at 4° C. with 0.1 ml of MOG 35-55 diluted in 0.1 M NaHCO3 buffer pH 9.5 at a concentration of 0.010 mg/ml. The plates were blocked with PBS 3% BSA for 2 hours. Samples were diluted in blocking buffer at 1:100 for IgG1 and IgG2a ELISA and incubated for 2 hours at room temperature. Antibody binding was tested by the addiction of alkaline phosphatase-conjugated monoclonal goat anti-mouse IgG1 and IgG2a (Southern Biotechnology Associates, Birmingham, Ala.), each at 1:1000 dilution in blocking buffer. Enzyme substrate was added and plates were read at 405 nm on a micro plate reader. Total IgE was measured by sandwich ELISA (PharMingen, San Diego, Calif.) following the manufacturer's instructions (see Spergel et al., J. Clin. Invest. 101:1614-1622, 1998).

EAE in Mice with a Disruption of the alpha-chain of FcγRIII (FcγRIII−/−) and of the γ chain Common to FcγRIII and FcεRI (FcR γ chain−/−): Myelin oligodendrocyte glycoprotein peptide (MOG) 3 5-55 was used to induce EAE (see Lock et al.) in mice with a disruption of the alpha chain of FcγRIII (FcγRIII−/−) (see Hazenbos et al., Immunity 5:181-188, 1996) and in mice with disruption of the γ chain common to FcγRIII and FcεRI (FcR γ chain−/−)(Takai et al., Cell 76:519-529, 1994) (see supra). We have previously shown that C57B1/6 mice (H-2b) immunized with MOG35-55 develop anaphylactic shock when re-exposed to this self-myelin peptide (Pedotti et al.). In order to explore the contribution of the Fc receptors to the development of anaphylaxis, we also challenged these two different strains of knockout mice with 0.1 mg of MOG35-55 i.p., 6 weeks after primary immunization, at a time when anaphylactic reactions to this peptide are known to occur (see id.).

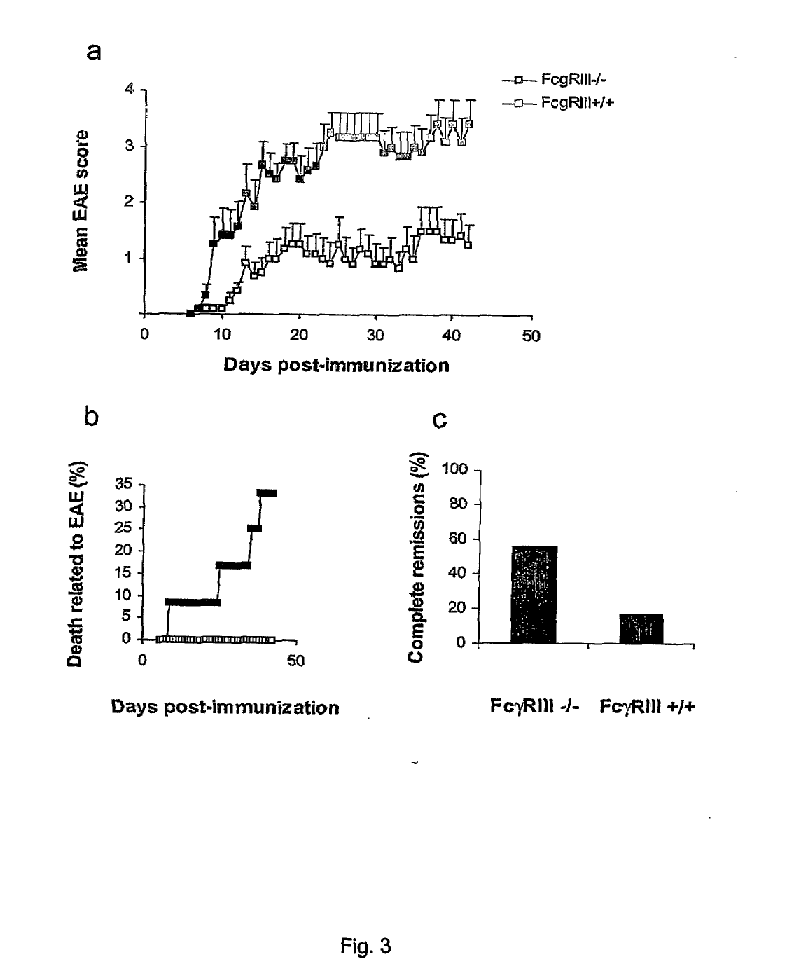

EAE was significantly ameliorated in mice lacking the low affinity IgG1 receptor FcγRIII (FIG. 3). For example, the incidence of EAE (9 of 12 in FcγRIII−/− vs 12 of 12 in +/+), mean peak of disease at day 15 (0.75±0.25 in FcγRIII−/− vs 2.67±0.43 in +/+; p=0.0035 by Mann-Whitney rank sum test), mean peak disease severity (2.42±0.61 in FcγRIII−/− vs 4.17±0.24 in +/+; p=0.0055 by t-test) and EAE related death (0 of 12 in FcγRIII−/− vs 4 of 12 in +/+) were significantly reduced in the knockout mice. The evaluation of the relapse/remission rate showed that the majority of FcγRIII−/− mice had more remissions when compared to the wild type animals, with the majority of EAE mice presenting periods of complete remission (5 of 9 in FcγRIII−/− vs 2 of 12 of the +/+). Histopathologic analysis revealed fewer inflammatory foci within the CNS of the knockout mice both in parenchyma and in meningi (4.2±1.9 vs 14.5±3 in the meninges of FcγRIII−/− vs +/+, p=0.0187 by t-test; 0.6±0.4 vs 5.5±1.2 in the parenchyma of FcγRIII−/− vs +/+, p=0.0036 by t-test), revealing that FcγRIII might be involved both in parenchyma and meningeal infiltration of inflammatory cells. FcγRIII−/− presented a lower incidence of anaphylaxis at challenge with MOG 35-55 compared to wild types (6 of 12 in FcγRIII−/− versus 7 of 8 in FcγRIII +/+), despite the higher titers of IgG1 and IgE observed in this group (Table 2). Since FcγRIII receptors are necessary for the expression of IgG1-mediated anaphylaxis (see Miyajima et al.), the presence of anaphylactic shock in mice lacking this receptor suggests that both IgG1 and IgE might mediate anaphylaxis to MOG35-55. Nevertheless, together with an impairment of other immune processes (see Hazenbos et al.), the abrogation of IgG1-mediated anaphylaxis is correlated with relative resistance to EAE in these mice.

TABLE 2
Serum antibody responses and allergic reactions to MOG35-55 in
FcγRIII −/−, in FcR γ chain −/− and their controls.

| | | | | Number |
|---|---|---|---|---|
| | | | | of mice |
| | | | | with |
| | | | | allergic |
| | Antibody responses¹ | | | reactions |
| | IgG1 | IgG2a | Total IgE | at |
| Strain | (O.D.) | (O.D.) | (μg/ml) | challenge² |
| FcγRIII −/− | 1.094 + 0.473 | 0.123 + 0.053 | 4.2 + 0.23 | 6/12 |
| Fcγ RIII +/+ | 0.259 + 0.047 | 0.048 + 0.021 | 1.45 + 0.47 | 7/8 |
| FcR | 1.343 + 0.506 | 0.140 + 0.074 | 4.1 + 0.17 | 0/11 |
| γ chain −/− | | | | |
| FcR | 0.434 + 0.179 | 0.071 + 0.022 | 3.45 + 0.51 | 5/9 |
| γ chain +/+ | | | | |

¹Serum from individual mice (5 to 12 per group) was collected 6 weeks after the induction of EAE and tested individually by ELISA assay. Numbers represent mean ± SEM.
²challenge was 6 weeks after the induction of EAE with MOG35-55 (0.1 mg) in PBS i.p., and presence of allergic reactions was confirmed by a reduction in body temperature of at least 0.5 degrees (see methods)

Amelioration of EAE was even more striking in mice lacking both FcγRIII and FcεRI (FcR γ chain−/−)(Table 3). In these mice, incidence of EAE (5 of 11 in FcR γchain−/− vs 12 of 12 in +/+; p=0.0046 by Fisher's exact test), mean disease severity at day 16 (0.64±0.39 in FcR γ chain−/− vs 2.42±0.4 in +/+; p=0.005 by Mann-Whitney rank sum test) and mean peak of disease severity (1.18±0.49 in FcR γ chain−/− vs 3.58±0.36 in +/+, p=0.0017 by Mann-Whitney rank sum test) were significantly reduced. All mice with deletion of these receptors had a remitting course (5 of 5 in FcR γ chain−/− vs 6 of 12 in +/+). Only 2 mice with deletion of both FcγRIII and FcεRI had one relapse, each, during the observation period of 6 weeks compared to the wild type mice where, of the mice surviving the acute phase (10 of 12), all had relapses. Histopathologic analysis revealed a paucity of CNS infiltrates in knockout mice compared to wild type mice (1.57±1.2 vs 45±13.6 in the meninges of FcR γ chain−/− vs +/+, p=0.0167 by Mann-Whitney rank sum test; 0.29±0.3 vs 46.3±22.8 in the parenchyma of FcR γ chain−/− vs +/+, p=0.0267 by Mann-Whitney rank sum test). Moreover, FcR γ chain−/− mice were completely protected against anaphylactic shock to MOG 35-55 (Table 2), while 56% (5 of 9) wild type mice had anaphylactic reactions.

TABLE 3
EAE in FcR γ chain −/− and +/+ mice.

| | | | | Peak | | |
|---|---|---|---|---|---|---|
| | Incidence | EAE onset | EAE score | disease | | Complete |
| Strain | (%) | (day)^{a} | (day 16)^{a} | severity^{a} | Death rate | remissions (%) |
| FcR γ chain −/− | 45% (5/11)^{b} | 11 ± 0.5 | 0.64 ± 0.4^{c} | 1.18 ± 0.5^{d} | 0% (0/11) | 100% (5/5) |
| FcR γ chain +/+ | 100% (12/12) | 12.4 ± 0.6 | 1.92 ± 0.4 | 3.58 ± 0.4 | 25% (3/12) | 50% (6/12) |

^{a}Data shown as mean ± SEM values.
^{b}P = 0.0046 (Fisher' exact test),
^{c}P = 0.005 (Mann-Whitney) and
^{d}P = 0.0017 (Mann-Whitney).
All P values are in comparison with the FcR γ chain +/+ group.

### Example 4

### Expression of Histamine Receptor H1 Preferentially on Myelin Specific Th1 T Cells

We explored the expression of some of the genes related to allergy in murine Th1 and Th2 T cell lines (TCL) activated against PLP 139-151.

Th1 and Th2 cell lines to PLP139-151: Th1 and Th2 T cell lines (TCL) were obtained as previously described in Garren et al., Immunity 15:15-22, 2001. For quantitative PCR analysis, TCL were harvested one week after stimulation with γ-irradiated spleen cells and PLP139-151. RNA was isolated using a Stratagene microRNA isolation kit (Stratagene, La Jolla, Calif.) according to manufacturers instructions.

Expression of Histamine Receptor 1: Compared to Th2 cells, encephalitogenic Th1 cells showed increased levels of transcripts for histamine type 1 receptor (H1R) (16 fold increase in Th1 vs Th2; p=0.009 by ANOVA), whereas Th2 cells showed increased transcripts of histamine type 2 receptor (H2R) (3 fold increase in Th2 vs Th1; p=0.004 by ANOVA, FIG. 1B).

### Example 5

### Immunohistochemical Detection of H1R and H2R in EAE Lesions

We analyzed the expression of H1R and H2R during EAE by immunohistochemistry, using two polyclonal antibodies generated in rabbits against the extracellular domain of these receptors.

Preparation of Tissue Samples for Histological Evaluation: for Histamine receptor detection in EAE brains, immunohistochemistry was performed as described with rabbit polyclonal antibodies (Rockland, Gilbertville, Pa.) generated against the extracellular domain (amino-terminal) peptides of H1R (SSASEDKMCEGN) (SEQ ID NO: 13) and H2R (SCCLDSIALKVT) (SEQ ID NO: 14). After mice were euthanized and perfused with cold PBS, tissues were embedded in OCT and quick-frozen. 4-6 micron cryostat sections were fixed with acetone. Staining with anti-H1R and -H2R antibodies at a 1:500 dilution was performed as described in Chabas et al. using avidin-biotin immunoperoxidase reagents (Vector Laboratories, Burlingame, Calif.). Sections were counterstained with hematoxylin.

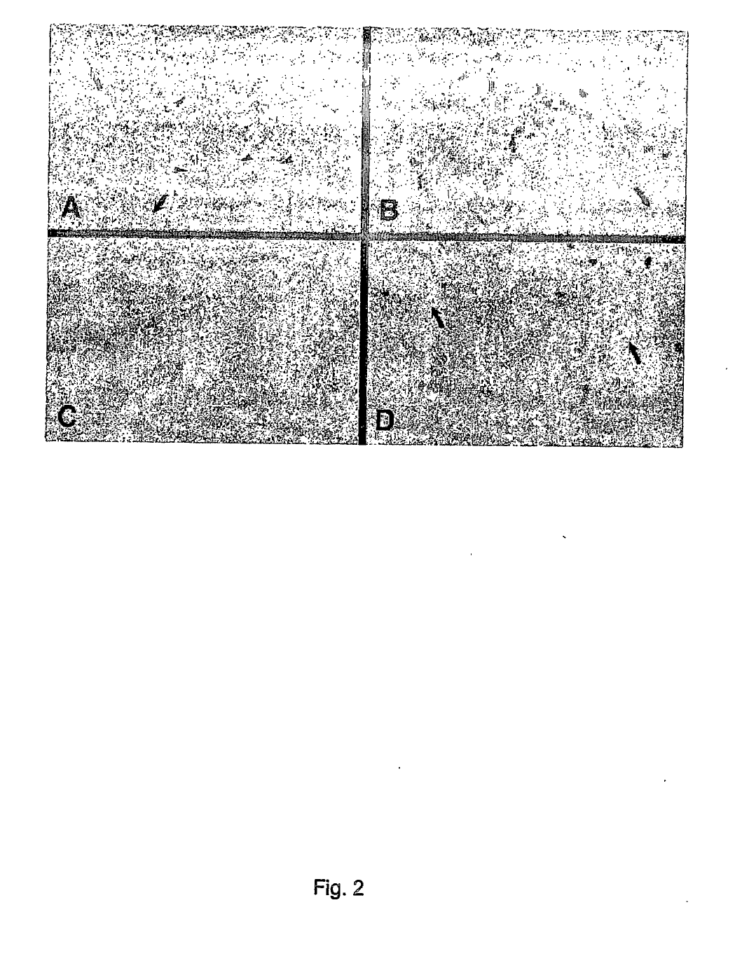

Expression of H1R and H2R: In naïve SJL mouse brain, H1R and H2R are expressed, as previously described (see Fukui et al., Agents Actions Suppl. 33:161-180, 1991; Arbones et al., Brain Res. 450:144-152, 1988; Hosli et al., Neurosci. Lett. 48:287-291, 1984; Karlstedt et al., J. Cereb. Blood Flow Metab. 19:321-330, 1999; Karnushima et al., J. Neurochem. 34:1201-1208, 1980), on rare astrocytes and on epithelial cells of the choroid plexus, while H2R was preferentially expressed on the endothelial cells of the blood vessels. In brains obtained from mice with EAE, H1R and H2R are expressed on the surface of mononuclear and other cells in the lesions (FIG. 2), revealing, for the first time, specific expression of these receptors in the inflammatory EAE infiltrates themselves.

### Example 6

### Modulation of EAE with Histamine 1 Receptor Blockade and PAFR Blockade

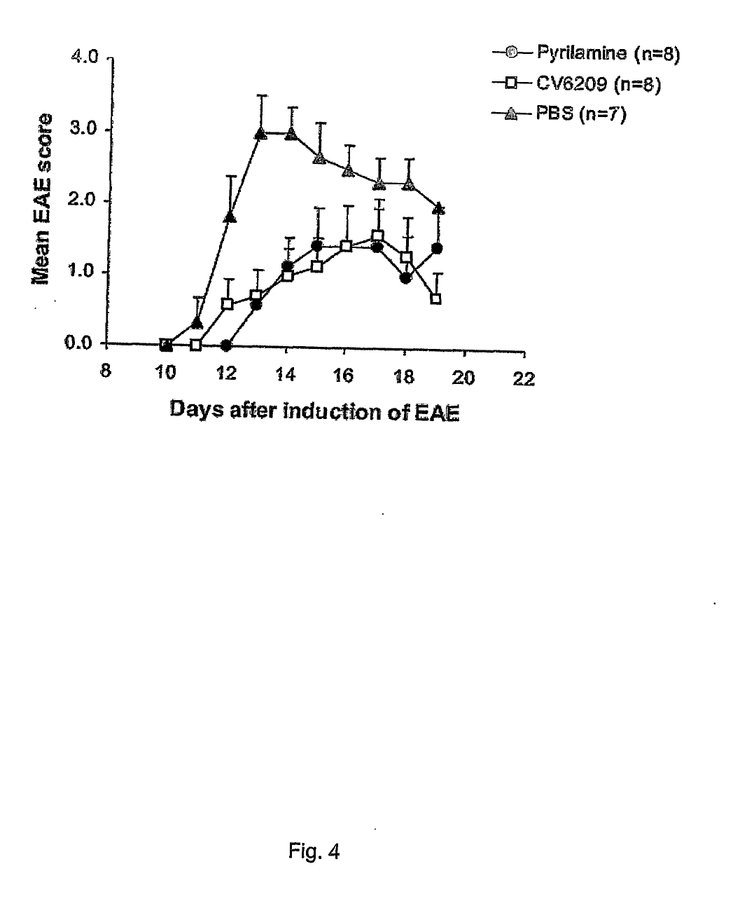

We then tested the functional roles of H1R and PAF in EAE. We targeted pharmacologically PAF and histamine, the main vasoactive mediators of murine anaphylaxis, and evaluated the development of EAE. EAE was induced in SJL (H-2s) mice with PLP139-151 and on the second day after the induction of the disease we started a daily i.p. treatment with the PAFR antagonist CV 6209, or with the H1R antagonist pyrilamine. CV 6209 has been previously used to block anaphylaxis in mice (see Strait et al.; Terashita et al., J. Pharmacol. Exp. Ther. 242:263-268, 1987). Treatment with either of these drugs ameliorated EAE (on day 13, mean EAE score was 0.57±0.2 in the pyrilamine treated group, 0.71±0.36 in the CV 6209 treated group, and 3±0.52 in the vehicle treated group; p=0.007 and p=0.0034 by t-test for pyrilamine and CV 6209, respectively, vs vehicle) (FIG. 4), suggesting a role for these two mediators in the development of EAE.

The previous examples are provided to illustrate but not to limit the scope of the claimed invention. Other variants of the inventions will be readily apparent to those of ordinary skill in the art and encompassed by the appended claims. All publications, patents, patent applications and other references cited herein are hereby incorporated by reference.

What is claimed is:

1. A method for treating or preventing an autoimmune disease in a subject, the method comprising:
administering to the subject an effective amount of an agent that blocks histamine H1 receptor (HIR), wherein the agent is not cyproheptadine or hydroxyzine.

2. The method of claim 1, wherein the agent is an H1-antihistamine.

3. The method of claim 2, wherein the H1-antihistamine is selected from the group consisting of an alkylamine, an ethanolamine, an ethylenediamine, a phenothiazine, a piperidine, and a piperazine.

4. The method of claim 3, wherein the H1-antihistamine is an ethylenediamine.

5. The method of claim 4, wherein the ethylenediamine is pyrilamine.

6. The method of claim 2, wherein the H1-antihistamine is a first generation H1-antihistamine.

7. The method of claim 6, wherein the first generation H1-antihistamine is pyrilamine.

8. The method of claim 1, wherein the autoimmune disease is Th1-mediated.

9. The method of claim 8, wherein the Th1-mediated autoimmune disease is an autoimmune demyelinating disease.

10. The method of claim 9, wherein the autoimmune demyelinating disease is multiple sclerosis.

11. The method of claim 9, wherein the agent is an H1-antihistamine not having a carboxylate group.

12. The method of claim 1, wherein the autoimmune disease is selected from the group consisting of rheumatoid arthritis, graft-versus host disease (GvHD), inflammatory bowel disease (IBD), insulin dependent diabetes mellitus (IDDM), multiple sclerosis, primary biliary cirrhosis, systemic sclerosis, psoriasis, autoimmune thyroiditis, and autoimmune thrombocytopenic purpura.

13. The method of claim 1, wherein the agent is administered by a route selected from the group consisting of intramuscular, subcutaneous, intravenous, parenteral, intranasal, intrapulmonary, and oral routes of administration.

14. The method of claim 1, wherein the H1R-blocking agent is not co-administered with a second active agent selected from the group consisting of dithiocarbamate disulfide derivatives, substituted 1,4-dihydropyridine bradykinin antagonists, heteroaryl substituted 1,4-dihydropyridine bradykinin antagonists, LTB-receptor antagonists comprising disubstituted phenyl-benzamidine derivatives, and small molecule antagonists of chemokine receptor CCR1.

15. The method of claim 1, wherein the agent does not substantially block serotonin receptor or mast cell biogenic amine secretion.

16. The method of claim 15, wherein the ED₅₀ dose for inhibition of the serotonin receptor by the agent is at least about 0.5 mg/kg.

17. The method of claim 16, wherein the ED₅₀ dose for inhibition of the serotonin receptor by the agent is at least about 0.6 mg/kg.

18. The method of claim 17, wherein the ED₅₀ dose for inhibition of the serotonin receptor by the agent is at least about 0.8 mg/kg.

19. The method of claim 1, wherein the subject does not have a second disease or disorder that requires treatment with the H1R-blocking agent.

20. The method of claim 1, wherein the subject has been diagnosed with an autoimmune disease.

21. The method of claim 1, further comprising monitoring the subject for a change in a symptom of the autoimmune disease.

22. The method of claim 1, wherein the H1R-blocking agent is co-administered with a second active agent.

23. The method of claim 22, wherein the second active agent is selected from the group consisting of
(a) a self-vector comprising a polynucleotide encoding a self-polypeptide associated with the disease;
(b) an immunomodulatory protein; and
(c) a vector encoding (b).

24. The method of claim 23, wherein the self-vector and the vector encoding an immunomodulatory protein are co-administered.

25. The method of claim 23, wherein the second active agent is the self-vector and further comprising co-administration of an immune modulatory sequence.

26. The method of claim 25, wherein the immune modulatory sequence is selected from the group consisting of
(a) 5′-Purine-Pyrimidine-[X]-[Y]-Pyrimidine-Pyrimidine-3′ and
(b) 5′-Purine-Purine-[X]-[Y]-Pyrimidine-Pyrimidine-3′,
wherein X and Y are any naturally occurring or synthetic nucleotide, except that X and Y cannot be cytosine-guanine.

27. The method of claim 23, wherein the immunomodulatory protein is a cytokine or a chemokine.

28. The method of claim 27, wherein the cytokine is selected from the group consisting of IL-4, IL-10, and IL-13.

29. The method of claim 1, wherein the autoimmune disease is a relapsing-remitting form of the disease.

30. The method of claim 29, wherein the administration of the agent decreases the relapse rate of the disease.

31. A method for treating or preventing multiple sclerosis in a subject, the method comprising:
administering to the subject an effective amount of an agent that blocks histamine H1 receptor (HIR), wherein the agent is not cyproheptadine or hydroxyzine

32. A method for treating or preventing multiple sclerosis in a subject, the method comprising:
administering to the subject an effective amount of an agent that blocks histamine 1 receptor (HIR), wherein the agent does not substantially block serotonin receptor or mast cell biogenic amine secretion.

33. A method for treating or preventing multiple sclerosis in a subject, the method comprising:
administering to the subject an effective amount of an agent that blocks histamine H1 receptor (HIR), wherein the agent does not substantially block serotonin receptor or mast cell biogenic amine secretion and is not co-administered with a second active agent.

34. A method for treating or preventing an autoimmune disease in a subject, the method comprising:
co-administering to the subject effective amounts of
(a) an agent that blocks histamine H1 receptor (H1R) and
(b) a second active agent.

35. The method of claim 34, wherein the second active agent is not an agent selected from the group consisting of dithiocarbamate disulfide derivatives, substituted 1,4-dihydropyridine bradykinin antagonists, heteroaryl substituted 1,4-dihydropyridine bradykinin antagonists, LTB-receptor antagonists comprising disubstituted phenyl-benzamidine derivatives, and small molecule antagonists of chemokine receptor CCR1.

36. The method of claim 34, wherein the second active agent is selected from the group consisting of
(a) a self-vector comprising a polynucleotide encoding a self-polypeptide associated with the disease;
(b) an immunomodulatory protein; and
(c) a vector encoding (b).

37. The method of claim 36, wherein the autoimmune disease is multiple sclerosis and the self-polypeptide is selected from the group consisting of myelin basic protein (MBP), proteolipid protein (PLP), myelin associated glycoprotein (MAG), cyclic nucleotide phosphodiesterase (CNPase), myelin-associated oligodendrocytic basic protein (MBOP), myelin oligodendrocyte protein (MOG), and alpha-B crystalline.

38. The method of claim 36, wherein the autoimmune disease is insulin dependent diabetes mellitus and the self-polypeptide is selected from the group consisting of insulin, insulin B chain, preproinsulin, proinsulin, glutamic acid decarboxylase 65 kDa and 67 kDa forms, tyrosine phosphatase IA2 or IA-2b, carboxypeptidase H, heat shock proteins, glima 38, islet cell antigen 69 kDa, p52, and islet cell glucose transporter (GLUT 2).

39. The method of claim 36, wherein the self-vector and the vector encoding the immunomodulatory protein are co-administered.

40. The method of claim 36, wherein the second active agent is the self-vector and further comprising the administration of an immune modulatory sequence.

41. The method of claim 40, wherein the immune modulatory sequence is selected from the group consisting of
(a) 5′-Purine-Pyrimidine-[X]-[Y]-Pyrimidine-Pyrimidine-3′ and
(b) 5′-Purine-Purine-[X]-[Y]-Pyrimidine-Pyrimidine-3′,
wherein X and Y are any naturally occurring or synthetic nucleotide, except that X and Y cannot be cytosine-guanine.

42. The method of claim 36, wherein the immunomodulatory protein is a cytokine or chemokine.

43. The method of claim 42, wherein the cytokine is selected from the group consisting of IL-4, IL-10, and IL-13.

44. The method of claim 34, wherein the autoimmune disease is a relapsing-remitting form of the disease.

45. The method of claim 44, wherein the administration of the agent decreases the relapse rate of the disease.

46. The method of claim 44, wherein the relapsing-remitting autoimmune disease is a relapsing-remitting form of multiple sclerosis.

47. A method for treating or preventing an autoimmune disease in a subject, the method comprising:
co-administering to the subject effective amounts of
(a) a self-vector comprising a polynucleotide encoding a self-polypeptide associated with the disease and
(b) a means for blocking histamine H1 receptor (H1R).

48. The method of claim 47, wherein the H1R-blocking means is pyrilamine.

49. The method of claim 47, wherein the autoimmune disease is multiple sclerosis.
